(19) United States
(12) Patent Application Publication
(10) Pub. No.: US 2007/0224674 A1
(43) Pub. Date: Sep. 27, 2007

(54) Selective Enrichiment of Microorganisms for Desired Metabolic Properties

(75) Inventors: John Stephen Bridger, Balwyn North Victoria (AU); Gunseli Ocal, Balwyn North Victoria (AU); Geoffrey John Dumsday, Vermont South Victoria (AU); Michael Zachariou, Vermont South Victoria (AU)

Correspondence Address: LADAS & PARRY, 26 WEST 61ST STREET, NEW YORK, NY, 10023, US

(73) Assignee: COMMONWEALTH SCIENTIFIC AND INDUSTRIAL RESEARCH OR, Campbell (AU)

(21) Appl. No.: 10/578,454

(22) PCT Filed: Nov. 15, 2004
(86) PCT No.: PCT/AU04/01577

§ 371 Date: Feb. 8, 2007

(30) Foreign Application Priority Data
Nov. 14, 2003 (AU) 2003906290

(51) Int. Cl. C12N 1/26; C12N 1/00; C12N 1/32; C12N 1/38

(52) U.S. Cl. 435/248; 435/243; 435/244; 435/247

(57) ABSTRACT

A method for selectively enriching for a microorganism able to metabolise a test substrate, and/or the enrichment of an enzyme involved in the metabolism of the test substrate, the method comprising the steps of: a) providing a population of microorganisms in a vessel, b) feeding fluid into the vessel at a controlled flow rate commencing with an initial flow rate, the fluid comprising a nutrient medium and, for at least part of the feed period, the test substrate, c) producing a signal indicative of the level of a metabolism indicator over the time-frame of the enrichment, and d) providing an output based on the signal to enable assessment of selective enrichment of a microorganism that metabolises the test substrate, and/or the enrichment of an enzyme produced by the microorganism that is involved in the metabolism of the test substrate. To accelerate the enrichment process, conditions may be set to increase the flow rate in stages as a steady state is detected. This may be achieved by presetting conditions to be met by the signal output to result in a change in the fluid flow rate, and changing the flow rate at which fluid is fed into the vessel when the conditions are met, wherein the preset conditions are a combination of a predetermined period of time and a preset value range within which the signal must remain for the predetermined period of time.

### RELATED APPLICATIONS

The present application claims priority from AU 2003906290, the entire contents of which are incorporated by reference.

### FIELD OF THE INVENTION

The present invention relates to a method for microbe and/or enzyme discovery. In particular, the present invention relates to a method for selectively enriching and thereby discovering a microorganism which can metabolise a test substrate. The present invention also enables the discovery of enzymes produced by a microorganism involved in the metabolism of a test substrate.

### BACKGROUND OF THE INVENTION

Techniques such as passaging in batch culture are still used today for the discovery of microorganisms which can metabolise a test substrate. These techniques are often labour intensive, slow and the expected outcome is not known until the enriched microbial population is plated onto selective media. Traditional methods for monitoring the activity or growth of a microbial population include measurement of biomass concentration and/or measurement of substrate consumption. These analytical techniques do not provide an assessment of the status of a microbial population in real-time to enable the status of the microbial culture to be determined, and intervention to occur if necessary.

The chemostat provides continuous culture and has been used for enrichment to facilitate the discovery of microorganisms with useful properties and the study of evolutionary pathways. The effectiveness of conventional continuous culture is limited because the status of the discovery process cannot be evaluated rapidly. In a limited number of cases carbon dioxide production and oxygen consumption have been used to monitor a continuous culture. However these techniques have been subject to a range of limitations imposed by the small number of applications to which the techniques have been considered applicable, and/or limitations based on deficiencies in the apparatus. For one example, off-line analysis of biomass concentration or residual substrate concentration is commonly required to evaluate the status of an enrichment process. Off-line analysis is time consuming in terms of the slow analytical techniques involved, and indeed the delays in developing an appropriate analytical procedure for determining analyte concentration. Furthermore, a significant level of infrastructure and staff trained in the use of the analytical equipment are also required.

Therefore, the applicants have identified a need for faster methods for microbial discovery.

### SUMMARY OF THE INVENTION

Accordingly, the present invention provides a method of selectively enriching for a microorganism able to metabolise a test substrate, and/or the enrichment of an enzyme involved in the metabolism of the test substrate, the method comprising the steps of

a) providing a population of microorganisms in a vessel,

b) feeding fluid into the vessel at a controlled flow rate commencing with an initial flow rate, the fluid comprising a nutrient medium and, for at least part of the feed period, the test substrate,

c) producing a signal indicative of the level of a metabolism indicator over the time-frame of the enrichment, and

d) providing an output based on the signal to enable assessment of selective enrichment of a microorganism that metabolises the test substrate, and/or the enrichment of an enzyme produced by the microorganism that is involved in the metabolism of the first substrate.

Where the microorganism produces an enzyme, or enzymes, that are involved in the metabolism of the test substrate, the method enables the selective enrichment of a microorganism that produces such enzyme or enzymes.

The present inventors have found that the above method for “on-line” determination of a change in the level of a metabolism indicator, such as O², as an indicator of cellular activity enables indirect measurement of biomass or substrate utilisation and have identified that this can be used to evaluate the status of a population of microorganisms in real-time. The inventors have further tailored this technique for enriching microorganisms that are capable of metabolising a test substrate, such as a hydrocarbon compound for which a microorganism is desired to be found to convert the compound (test substrate) into a different hydrocarbon(s) and/or break the compound down with water as a byproduct. Such metabolism may be accompanied by the production, or up-regulation of an enzyme or enzymes that are involved in the metabolism of the test substrate. Thus, the metabolism of the microorganism also reflects an increase in the population or amount of enzyme in the vessel (compared to the relative amount of that enzyme in the vessel at the outset of the procedure) that has the desired function of catalysing the reaction of the test substrate.

The technique developed by the inventors has further advantages in terms of its flexibility in discovering microorganisms capable of metabolising a test substrate in conditions selected by the operator (i.e. a selective pressure), and potentially modified by the operator over time. The modification of conditions can be used to identify microorganisms that have the capability of producing an enzyme or enzymes that assist in the metabolism of the test substrate under such conditions. This is of particular assistance in the identification of microorganisms (and consequently, optionally, enzymes) that are involved in the metabolism of substrates in harsh or challenging conditions. All of this is evaluated in real-time without the need to separately measure substrate levels or determine biomass concentration.

In a preferred embodiment, the method further comprises presetting conditions to be met by the signal output to result in a change in the fluid flow rate, and changing the flow rate at which fluid is fed into the vessel when the conditions are met, wherein the preset conditions are a combination of a predetermined period of time and a preset value range within which the signal must remain for the predetermined period of time.

The flow rate of the fluid fed into the vessel is suitably increased from the initial flow rate on meeting the preset conditions to reduce the hydraulic retention time, and thereby increase selectivity for a microorganism that metabolises the test substrate. Increasing the flow rate of the fluid fed into the vessel will facilitate the selective enrichment of microorganisms which metabolise the test substrate more quickly and therefore reproduce more quickly. In effect, the preset conditions should be set to define the maintenance of steady state in the culture over the predetermined time period. The predetermined time period may be in a time unit of measurement (eg a number of minutes or hours), or may be set by reference to a predetermined multiple (including fractions) of the hydraulic retention time of the vessel. Consequently it will be understood that the reference to a predetermined time period need not be an exact, repeated number of hours, especially if the fluid flow rate is changed over time.

The flow rate of the fluid fed into the vessel may be increased by increasing the flow rate of the test substrate. Further, the fluid flow rate may be increased by increasing the flow rate of the nutrient medium in addition to the test substrate. If the level of test substrate in the vessel is sufficiently high it is possible for the flow rate to be increased by increasing the flow rate of the nutrient medium alone, although this is not preferred. Where the flow rate of both the test substrate and the nutrient medium is increased, it is convenient for the flow rates to be increased proportionally such that the concentration of the test substrate in the fluid fed into the vessel remains substantially constant.

The metabolism indicator used in the method of the invention may be the uptake or release of a molecule involved in metabolism of the test substrate. Generally, such molecules are electron acceptors. These are described in further detail in the Examples. Examples of the metabolism indicator are oxygen, carbon dioxide, carbonate, sulphur, sulphate, nitrate, fumarate and iron. Others are also known. According to one specific embodiment, the metabolism indicator is selected from oxygen, sulphate, sulphur, nitrate, fumarate and iron.

The signal of the level of the metabolism indicator is preferably provided as a visual output, such as a plot of points which represent the level of the metabolism indicator against time. The signal output will be an electrical signal, and therefore the plot may be of the electrical output (eg current) against time. Otherwise, in the example of the metabolism indicator being oxygen uptake, the electrical signal may be converted into oxygen concentration or oxygen uptake rate, and this may be plotted against time. The output could also be a numerical digital or liquid crystal display. The visual output may conveniently be updated in periods of less than 20 minutes. Ideally, the visual output is updated in periods of 10 mins or less.

As a consequence of this, in the embodiment where conditions are pre-set to result in a change in the fluid flow rate, the values set may be in units of the direct signal value, or indirectly by reference to the level of the metabolism indicator, or any other related unit of measurement.

In most situations, a controller will be set to increase the flow of nutrient medium and/or test substrate into the vessel in response to the signal meeting the preset conditions. This particularly enables the selecting of microorganisms that metabolise the test substrate and reproduce quickly, as microorganisms not able to reproduce quickly enough will be washed out of the apparatus. Thus, according to one embodiment, the supply mechanism operates to supply the nutrient medium and the test substrate to the vessel at an initial flow rate, and the controller is set to increase the flow rate from the initial flow rate in response to the signal meeting the preset conditions. However, it is appreciated by the applicants that decreases could be set, especially in a later stage of an operation being conducted on the apparatus.

Generally, the intention of pre-setting the range (upper and lower signal ranges) of the signal is to identify when the culture has reached a steady-state. Once a steady state has been identified, it is possible to change the flow of fluid (nutrient medium and/or test substrate) into the vessel.

The fluids fed into the vessel are most conveniently fed in through separate feed or supply mechanisms. Being able to supply the two fluids separately offers more control to the user in terms of modifying the conditions under which the microorganisms are required to metabolise and reproduce. Secondly, this offers advantages in terms of switching from one test substrate to the next without changing the nutrient medium fed into the vessel.

Preferably the preset range of the signal is set by the user. In the case where the signal is representative of the level of oxygen in the vessel, the user preferably selects the maximum and minimum levels in any appropriate unit of measurement, such as mg of oxygen per ml of liquid in the vessel, biological oxygen demand (BOD), oxygen uptake rate (OUR) or similar. Of course, where the metabolism indicator being detected is another indicator such as carbon dioxide, nitrate, iron or so forth, the user suitably selects the maximum and minimum levels in the unit of measurement relevant to those signals.

Preferably, the user also sets the predetermined time period.

Preferably, the user also sets the pH level and temperature of the vessel. As will be understood, this then enables user to modify the conditions to select a microorganism able to metabolise the test substrate in specific conditions (eg high or low pH; high or low temperature etc), or an associated enzyme. These conditions can be set at levels that impose a selective pressure (in addition to the pressure of the test substrate) on the contents of the vessel to select for a microorganism and/or enzyme that tolerates or utilises the selective pressure. Possible selective pressures are an increase or decrease in temperature, pH, aeration, dissolved gas content, salt concentration, and the presence or absence of a chemical compound such as a toxin or nutrient component.

The user may further be able to set other conditions that impact on the metabolism, such as the oxygen level or aeration rate.

The population of microorganisms used in the method of the invention may be a heterogeneous population, such as activated sludge, or may be a homogeneous population.

Preferably the population of microorganisms is a heterogeneous population. It may in this case be a heterogeneous population containing at least 10, preferably 100 different strains or species of microorganism. This is explained further in the detailed description.

The method of the invention may further comprise the step of subjecting the population of microorganisms to a mutagen, such as a chemical mutagen or ultra-violet light.

The method of the invention may further comprise the step of isolating the enriched microorganism.

The present invention further provides a microorganism when enriched or isolated by the method described above.

The invention also provides for a corresponding method for assessing the selective enrichment over the timeframe of the enrichment process, which includes steps (a) to (d) as outlined above.

### BRIEF DESCRIPTION OF THE DRAWINGS

FIG. 1 is a schematic illustration of the apparatus of one embodiment of the invention.

FIG. 2 is a schematic illustration of the apparatus of FIG. 1 with further apparatus components.

FIG. 3 shows the correlation between OUR and microbial activity as determined by conventional analytical techniques, as well as the correlation between different conventional analyses, using acetic acid as the test substrate.

FIG. 4 shows the correlation between OUR and microbial activity as determined by conventional analytical techniques, as well as the correlation between different conventional analyses, using sodium acetate as the test substrate.

FIG. 5 shows the correlation between OUR and microbial activity as determined by conventional analytical techniques, as well as the correlation between different conventional analyses, using benzyl alcohol as the test substrate.

FIG. 6 demonstrates the correlation between a population change and BOD—the BOD and residual substrate concentration.

FIG. 7 demonstrates the correlation between a population change and BOD—the changes to the population as measured using viable cell counts and optical density.

FIG. 8 shows the increase in BOD after the addition of 1-methyl-2-pyrrolidinone to a culture.

FIG. 9 shows BOD during growth of microorganisms from activated sludge on 1-methyl-2-pyrrolidinone.

FIG. 10 shows BOD output during growth of microorganisms from activated sludge using dodecane as the test substrate.

FIG. 11 shows the effect of flow rate on the BOD of a 1,3-propanediol-degrading microbial population.

FIG. 12 shows the optical density (OD) readings of samples taken from the vessel in Example 7 at different feed flow rates.

FIG. 13 is a graph of dilution rate against enzyme activity for the isolates described in Example 7.

FIG. 14 is a graph of the biological oxygen demand reading taken from the vessel over time in Example 8.1.

FIG. 15 is a micrograph of a sample taken at a late stage of operation of the method of the invention at 80° C. in accordance with Example 8.2.

FIG. 16 is a graph of relative nitrate concentration over time and pH over time for the contents of the vessel during population development in Example 9.

FIG. 17 is a graph of relative nitrate concentration over time and pH over time for the contents of the vessel over the full operation of Example 9.

FIG. 18 is a micrograph of a sample taken at a late stage of operation of the invention in accordance with Example 9.

### DETAILED DESCRIPTION OF PREFERRED EMBODIMENTS

The present invention provides a method for the selective enrichment of a microorganism able to metabolise a test substrate. It will be understood that a “microorganism” means any microorganism, for example, bacteria, fungi, yeast, protozoans, algae or viruses. Any of these microorganisms can be selectively enriched by designing the enrichment conditions to favour the growth of a microorganism with a particular characteristic. The microorganism may be an aerobic or anaerobic microorganism. Specific microorganisms in one or the other of these classes can be enriched by imposing the appropriate conditions for either aerobic respiration or anaerobic respiration to select for a microorganism in the chosen class.

An enzyme is a protein which catalyses a chemical reaction, such as a metabolic reaction. The enzyme may be directly or indirectly associated with the microorganism which produces the enzyme. For example, the enzyme may be non-covalently bound to the cell membrane of the microorganism, may be located in the cytoplasm of the microorganism, or may be one secreted from the cell into the surrounding medium.

Where the chemical reaction is a metabolic reaction, the enzyme is involved in the metabolism of a test substrate. As used herein, “involved” means that the enzyme catalyses a reaction which is part of a metabolic pathway. The enzyme may catalyse more than one reaction in the metabolic pathway, and may catalyse anabolic or catabolic reactions. Typically, the enzyme will catalyse at least the first reaction in a metabolic pathway.

It must be noted that as used herein, the singular forms “a”, “an”, and “the” include plural reference unless the context clearly dictates otherwise. Thus, for example, reference to a microorganism includes a plurality of microorganisms.

As used herein, the term “enrichment” means an increase in the number (or relative concentration) of microorganisms in a population which are able to metabolise the test substrate compared to microorganisms that do not metabolise the test substrate, or an increase in the number of molecules (or relative concentration) of the enzyme involved in metabolism of the test substrate compared with the starting enzyme population of the population of microorganisms.

In the case of enzymes, in addition to increasing the number of molecules of the enzyme in the vessel, the enzyme may be mutated over the time period of the enrichment to improve its properties in the conditions to which it is exposed in the vessel. Examples of the improved properties are increased catalytic rate, tolerance to a selective pressure (such as high temperature—i.e. thermal tolerance) or utilisation of the condition. Indeed, the method of the invention provides an excellent environment and feedback information to drive the enzymes towards such mutations.

In step (b) of the method, the feeding of fluid into the vessel drives or results in the selective enrichment of the microorganism (and/or enzyme) that metabolises the test substrate.

“Metabolise” means to use the test substrate in a chemical reaction within the microorganism by either catabolism or anabolism. Therefore a test substrate may be used in a chemical reaction that combines the test substrate into a more complex molecule, or may be used in a chemical reaction which breaks down the test substrate into a simple molecule.

The “test substrate” is any substrate for which it is desired to screen for a microorganism able to metabolise the test substrate and does not include substrates which are commonly metabolised, such as glucose and acetate. The purpose of the method of the invention is to arrive at a microorganism population that is able to metabolise the test substrate, and/or an enzyme associated with the metabolism. Generally the method is suited for the situation where a microorganism or enzyme is desired to be formed which has the ability to metabolise a new (test) substrate which no suitable microorganism is known to metabolise. Such test substrates may be environmental toxins, waste materials, undesired byproducts of a reaction.

The technique and the controls required are very different to techniques where the substrate is known to be a substrate for certain microorganisms, or is a common substrate for a large range of microorganisms. Typically, the method of the invention will be used to selectively enrich microorganisms which can metabolise an organic carbon-containing molecule. The term “organic carbon-containing molecule” refers to aliphatic and aromatic hydrocarbons and derivatives thereof, including carbohydrates other than commonly metabolised substrates such as glucose. Alternatively, the test substrate may be a sulphur-containing test substrate and/or a nitrogen-containing test substrate.

The method comprises the step of providing a population of microorganisms in a vessel.

It will be clearly understood that the population of microorganisms may be a homogeneous population of microorganisms or may be a heterogeneous population of microorganisms. A homogeneous population may be useful to selectively enrich for a microorganism by evolution. A homogeneous population is one which contains a single species, but which may be a phenotypically heterogeneous population before, during and/or after enrichment.

Where the population of microorganisms is a heterogeneous population this may be, for example, a microbial library or a heterogeneous population, such as activated sludge. A good diversity of the starting population of microorganisms, gives very good results in the method of the invention. Therefore, the heterogeneous population preferably comprises at least 10, preferably at least 100 different strains of microorganism. The heterogeneous population more preferably comprises at least 10, preferably at least 100 different species of microorganism, for increased diversity. The greater the diversity of the population, the better the anticipated results.

Activated sludge is the product that results when primary effluent of raw sewage is mixed with bacteria-laden sludge and then agitated and aerated to provide biological treatment in order to accelerate the breakdown of organic matter in the raw sewage undergoing secondary waste treatment. The present inventors have successfully used activated sludge as the starting microbial population in the method of the invention to enrich for microorganisms able to metabolise diverse test substrates under a diverse range of conditions. This population has over 100 different species (and over 100 strains) of microorganisms.

The fluid comprises a nutrient medium and the test substrate. A “nutrient medium” is a growth medium which comprises all of the nutrients required for growth of a microorganism but essentially no amount of the test substrate or substrates similar to (eg in the same class as) the test substrate. The concept of “similar substrates” to the test substrate is described below. The nutrient medium will depend upon the microbial population being enriched and the substrate being tested. However it is generally a nitrogen (ammonium), phosphorus, sulphur, salt (eg Na, Mg, Ca) and trace metal-containing solution. For example, when the method of the invention is used to enrich for microorganisms able to metabolise acetic acid (an organic carbon-containing substrate), the nutrient medium may be that set out in the Examples below. The nutrient medium may contain a trace amount of the similar substrate provided that the amount does not interfere with the detection of the enrichment process. The amount of the similar substrate must be such that it does not interfere with detection of the enrichment process. Ideally, the nutrient medium contains no similar substrates. For example, where the test substrate is an organic carbon-containing test substrate the nutrient medium contains substantially no organic carbon-containing material. There is also the possibility that the test substrate could be used as the sole source of another nutrient other than carbon, for example nitrogen or sulphur. In this case the nitrogen or sulphur would need to be eliminated from the nutrient medium or kept at a concentration that does not interfere with enrichment process.

“Similar substrate” means a substrate which the microorganism can metabolise as an alternative to the test substrate. For example, where the method is used to selectively enrich a microorganism able to catabolise a particular organic carbon-containing substrate, a similar substrate is an alternative carbon-containing substrate which the microorganism is able to catabolise. Where the test substrate is a small hydrocarbon molecule, “similar substrates” to be avoided in the nutrient medium are other small hydrocarbon (including carbohydrate) molecules, such as glucose and acetate.

The test substrate may be fed into the vessel as part of the nutrient medium or separately to the nutrient medium. For better control, these fluids can be fed into the vessel independently.

The initial flow rate at which the nutrient medium and test substrate are fed into the vessel, or hydraulic retention time, is chosen by reference to factors such as the starting population of microorganisms, the nutrient medium, the temperature of the vessel and the fluid, the pH of the fluid, and the stage of enrichment, and the vessel volume. Hydraulic retention time is a measure of the length of time that liquid remains in the vessel. It equals V/Q (V=vessel volume, Q=flow rate). Typically the initial hydraulic retention time will be relatively long in order to establish a steady state within the vessel. During feeding of the fluid into the vessel, there is also an outflow (or overflow) of fluid exiting the vessel, such that the fluid volume in the vessel remains constant.

The selective enrichment of a microorganism and/or enzyme is made possible through the on-line monitoring a signal representative of the level of metabolism indicator, and the real-time output based on the signal.

As used herein, “on-line” means that a reading of the level of metabolism indicator is taken directly from the contents of the vessel, be that the fluid in the vessel or gas in the headspace of the vessel, and is electronically converted into the output. Generally this means that the signal is taken and the output produced without direction or human involvement. The reading may be taken in the vessel itself or in a conduit through which contents of the vessel may flow.

The signal may be produced by a probe positioned to take readings from the contents of the vessel.

The purpose of this arrangement is to enable signal readings to be taken without removal of fluid from the apparatus, including the vessel and any associated conduits. Monitoring the level of a metabolism indicator on-line alleviates the need for off-line analyses in order to monitor enrichment and therefore facilitates the real-time determination of enrichment.

As used herein, “real-time” means that the output of the level of the metabolism indicator is provided fast enough to enable the status of the microbial culture in response to a change in conditions to be determined, and intervention to occur if necessary. An example of intervention provided by real-time monitoring is that which prevents the loss of a microbial population in response to a change in the conditions of the population that does not enable metabolism of the test substrate by a microorganism in the population. The frequency required to provide the output of the level of the metabolism indicator will depend upon the status of the enrichment process and the growth rate of the microorganism being enriched. The output of the level of the metabolism indicator should be updated in periods of 20 minutes or less, most suitably around 10 minutes or less.

The metabolism indicator may be any indicator of metabolism, for example a molecule consumed during metabolism such as oxygen, or a molecule produced by metabolism, provided only that the level of the metabolism indicator is able to be monitored on-line and used to provide an output of the level of the metabolism indicator. Examples of metabolism indicators identified as being capable of being monitored on-line with a probe are oxygen, carbon dioxide, carbonate, sulphate, sulphur, nitrate, fumarate and iron. These molecules act as terminal electron acceptors in the metabolism and the level of their presence in solution can be detected by a probe.

According to one embodiment, the oxygen uptake rate (OUR) of the microbial culture may be used as the metabolism indicator, particularly for the identification of aerobes. This can be determined by adding oxygen to the culture followed by the determination of a change in the oxygen level after a specific time period. The OUR gives a real-time measure of both substrate utilisation and growth of the population. By using this value to calculate the biological oxygen demand (BOD) of the test substrate in the fluid fed into the vessel, the level of substrate utilised can be determined. This is described further below in the examples.

Similar calculations can be used for any other metabolism indicator and signal or probe combination. For example, in the situation where the microbe is an anaerobe and does not use oxygen to respire during metabolism of the target molecule, but instead uses nitrate as the terminal electron acceptor, a nitrate probe can be used to monitor levels of nitrate.

The method of the invention may further comprise subjecting the microorganism population to a mutagen. As used herein, a mutagen is an agent which induces a change in the phenotype of a microorganism. A person skilled in the art will be readily able to determine a suitable mutagen, for example a chemical mutagen or an ultra-violet light with a wave length of 10 nm to 400 nm may be used.

The method of the invention may further comprise the step of discovering the enriched microorganism and/or enzyme. Discovery refers to isolation of the enriched microorganism and/or enzyme. This step may be readily performed by the person skilled in the art using standard microbiological techniques.

For example, where the enriched microorganism is a bacteria, a sample of the enriched culture may be plated onto solid nutrient medium which contains the test substrate, and the plate incubated under the conditions which enable enriched bacteria to metabolise the test substrate. Individual colonies formed by the enriched bacteria can then be isolated, and subjected to further characterisation steps if required.

Methods of isolating-enzymes from microorganisms are known in the art. The method used will depend upon the source of the enzyme, the enzyme to be isolated, and the purity in which the enzyme is required to be isolated.

A typical method of isolating an enzyme would include:

1) Preparation of crude extract, such as by cell lysis or membrane solubilisation;

2) An optional step of removal of nucleic acids, and/or ribosomes;

3) Precipitation with a precipitating agent such as (NH4)2SO4;

4) Purification, usually by chromatography such as one or more of affinity, gel filtration, ion-exchange, and hydroxyapatite chromatography; and

5) Removal of salt from the enzyme, for example by filtration.

This is one example of a method of isolating an enzyme from a microorganism, and it will be understood that any other methods known in the art could be used.

Where not otherwise described herein, the techniques employed in putting the invention into practice are conventional microbiological and chemical techniques known within the art. Such techniques are well known to the skilled worker, and are fully explained in the literature. See, eg., Bergey's Manual of Systematic Bacteriology; Bergey's Manual of Determinative Bacteriology; The Prokaryotes, Starr, Stolp, Truper, Balows, Schlegel, editors; Handbook of Microbiological Media, Atlas; Biology of Microorganisms, Brock, Madigan, Martinko and Parker; Methods for General and Molecular Bacteriology, Gerhardt, Murray, Wood, Krieg, editors.

The invention will now be described by way of the following non-limiting examples and drawings. Although any materials and methods similar or equivalent to those described herein can be used to practice or test the invention, the preferred materials and methods are now described.

### EXAMPLES

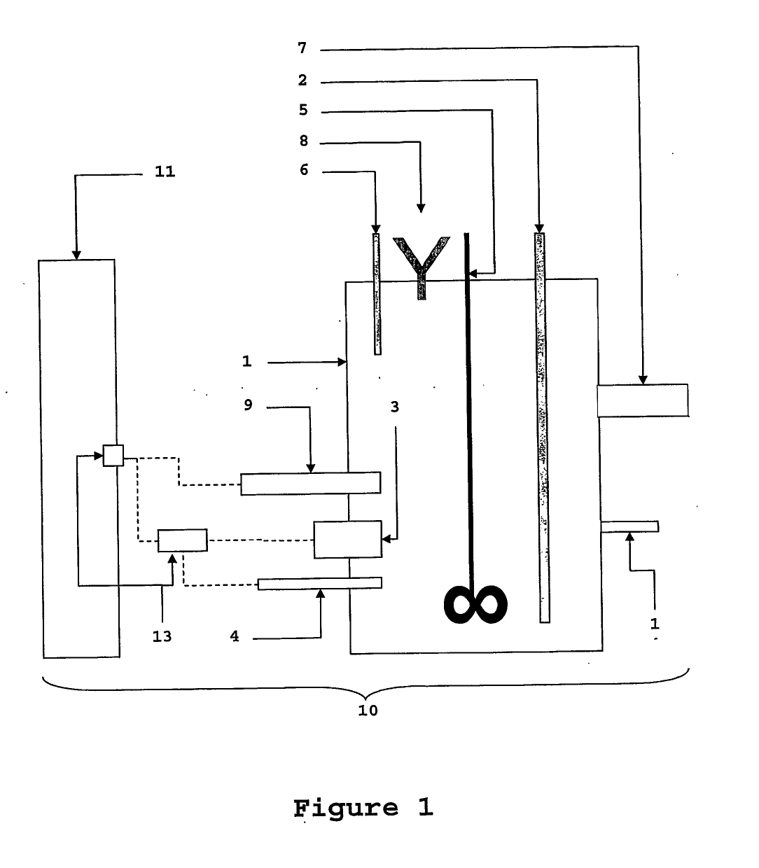

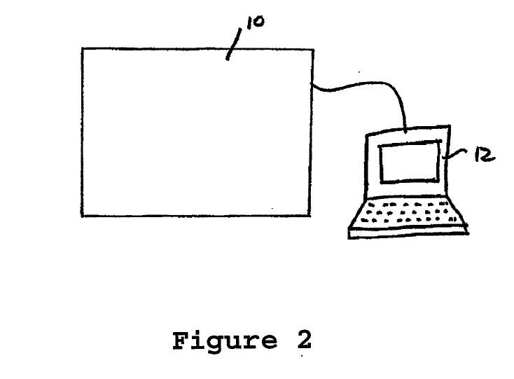

FIGS. 1 and 2 illustrate an example of the apparatus upon which the method of the present invention can be performed when the metabolism indicator is oxygen. Variations on the device for other metabolism indicators are set out in following Examples. The apparatus comprises a vessel or bioreactor 1 with an oxygen (air) injection means 2 and a dissolved oxygen measuring probe 3. The vessel is also associated with a temperature control means, including a temperature probe 4. The vessel also includes a stirrer 5 for stirring the contents of the vessel.

Fluid is fed into the vessel through inlet 6. The embodiment illustrated contains one inlet for feeding a combination of nutrient medium and test substrate, however separate inlets for each may be provided. A supply mechanism (not illustrated) controls flow of fluid into the vessel via inlet 6. The supply mechanism is connected to a nutrient medium supply well and a test substrate supply well (also not shown) to enable the control of the ratio of the two fluids, and the flow rate into the vessel 1. Overflow fluid is removed from the vessel via fluid outlet 7.

The apparatus further comprises an inlet 8 for the supply of acid and alkali for the control of pH in the vessel. Two inlets, are each for acid and base, can alternatively be used. The pH of the fluid in the vessel is measured by a pH probe 9.

Further components of the apparatus illustrated include electronics plugs 13 and a sample line/drain 14.

The apparatus may be provided as a unit 10 containing the elements described above, together with a control unit 11. The control unit 11 is under the control of a computer 12, which includes a monitor and a keyboard. The computer is programmed to provide a graphical user interface with the control program which allows the user to control the parameters described in the Examples that follow. The computer interacts with the control unit so that they together operate to control the supply mechanism to control the supply of fluids into the vessel in response to the probe signal.

The apparatus illustrated provides a series of visual outputs. This output shows the settings entered by the user for defining the pH, temperature, aeration level, upper and lower limits of the probe signal range (measured in this case in terms of the level of oxygen, measured in mg l⁻¹), the initial flow rate of inlet fluid, the flow increment (positive value represents increase), and the predetermined time period (which can be set as a number of vessel volumes).

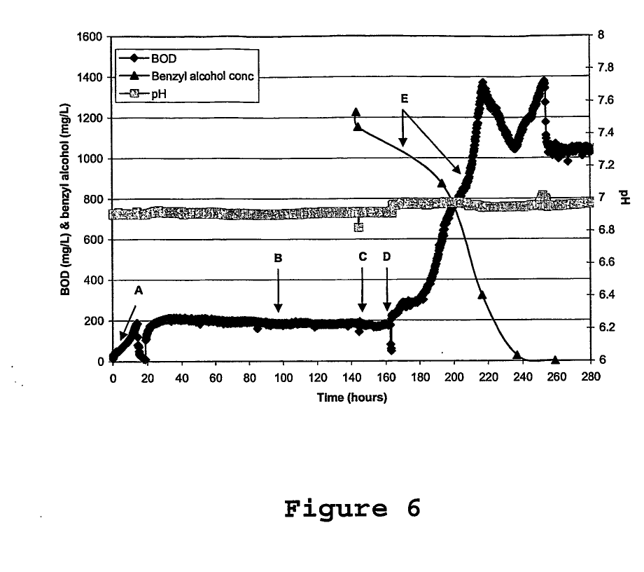

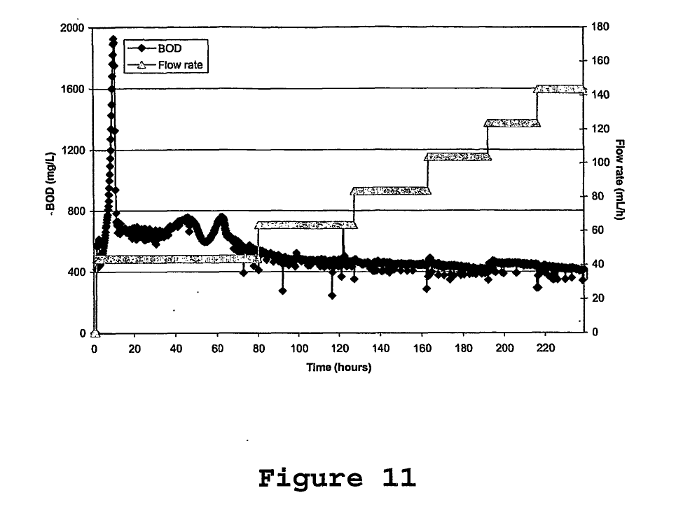

The screen can be switched to an output of one of a number of graphs including those illustrated (with entries) in FIGS. 6 to 11.

The mechanical and program components of the apparatus will be well understood to those skilled in the relevant arts, in the light of the functional description provided herein.

In the following examples, unless otherwise specified, the nutrient medium used was a defined medium (DM) prepared as outlined in the first section of Appendix 1.

### Example 1

### Correlation Between Oxygen Uptake Rate (OUR) and Microbial Activity

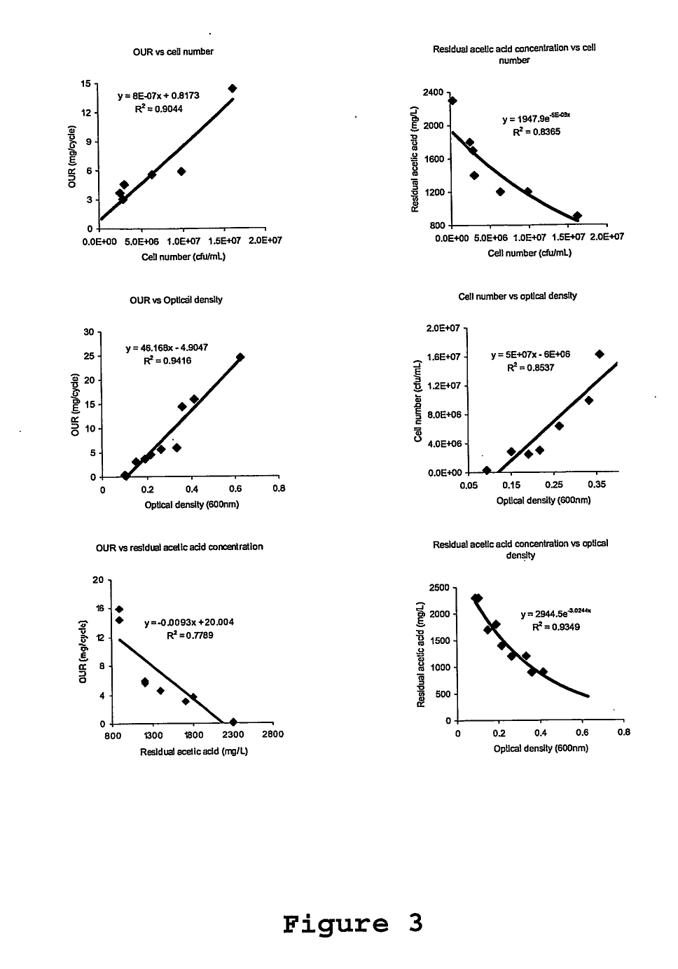

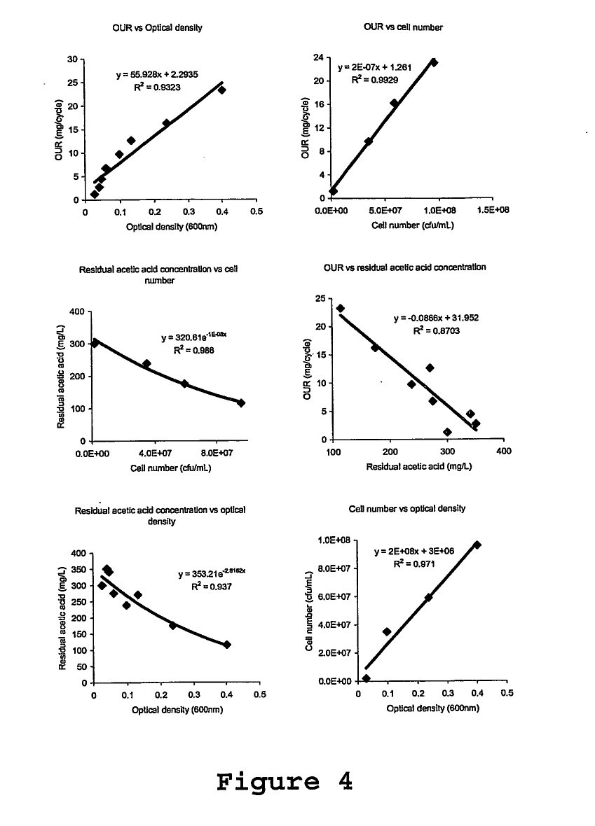

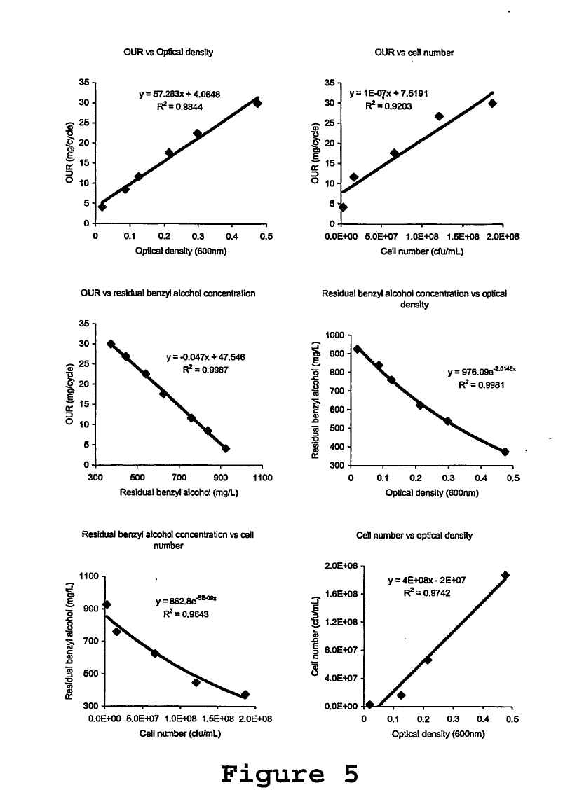

To determine whether the oxygen uptake rate (OUR) is a true reflection of the activity of a microbial population, OUR was compared with analytical techniques that are typically used to evaluate microbial activity. A 100 ml shake flask culture of Pseudomonas putida F1 (ATCC 70007) that had been grown for 48 hours at 28° C. shaking at 190 rpm, then centrifuged and resuspended in 10 ml of defined medium (DM) with no carbon source added, was used to inoculate DM that contained 1.5 or 2.0 g l⁻¹ acetic acid, or 1.0 g l⁻¹ benzyl alcohol. After inoculation the culture was sampled periodically for determination of microbial activity by conventional analytical techniques such as viable cell number, optical density (600 nm) and residual substrate concentration. OUR was measured every 10 minutes. These conventional analyses were compared with OUR measured using the method of the invention. The experiment was repeated three times, twice with acetic acid as the test substrate and once with benzyl alcohol. The correlated results of the three experiments are shown in FIGS. 3, 4 and 5.

From the data shown in FIGS. 3, 4 and 5 there is a clear linear correlation between OUR and both substrate consumption and biomass concentration regardless of whether the substrate is acetic acid or benzyl alcohol. The correlation between biomass and substrate utilisation shows a clear exponential correlation. This is probably because the yield value (Y_{x/g}; grams of biomass per gram of substrate) is not a true constant and is actually dependent on growth rate which is changing constantly during growth in batch culture (Mandelstam et al., Biochemistry of Bacterial Growth. 3rd Edition, Blackwell Scientific Publications, Oxford, UK, 1982). The method of the invention can therefore be used as a superior alternative to monitor the status of enrichment in real-time. This provides the operator with the opportunity to rapidly refine the culture conditions or determine the effect on a culture of changing the many parameters which can affect the enrichment of a microbial population.

### Example 2

### Demonstration of Real-Time Monitoring of a Population Change

To test and demonstrate the operation of the method of the invention, a control experiment was performed which compared the output of the method with off-line measurements that are traditionally used to monitor microbial activity. Techniques that are typically used include measurement of the residual substrate concentration and/or measurement of biomass concentration (viable count and optical density). These methods were compared with the output of the present method to demonstrate the utility of the method.

For these control experiments a steady state culture of an Escherichia coli BL21DE3 which was supplied by Novagen (Novagen Inc., Madison, Wis., USA) and was expected to grow on glucose only was used. The culture was established using 5 ml of an E. coli culture taken from a 100 ml shake flask culture which had been grown for 17 hours shaking at 200 rpm and 30° C. in defined medium with 1.0 g l⁻¹ glucose as the carbon source. Although the feed contained another substrate (benzyl alcohol), no growth on this substrate was expected because this microbial population was known to be unable to use this carbon source for growth. When steady state had been established, 10 mL of a 100 mL shake flask culture of Pseudomonas putida F1 was added. The P. putida F1 culture had been grown at 30° C. for 17 hours, with shaking at 200 rpm, in defined medium with 1.0 g l⁻¹ glucose as the carbon source. The P. putida F1 was supplied by the American Type Culture Collection (ATCC) and was expected to grow on benzyl alcohol and/or glucose. The OUR was expected to change as a result of the increased microbial activity after the addition of P. putida.

2.1 Growth of E. coli and P. putida on and in the Presence of Glucose and Benzyl Alcohol in Defined Medium in Batch Culture

The success of this control experiment was dependent upon the ability of E. coli to grow on glucose in DM and grow in DM in the presence of benzyl alcohol (i.e. benzyl alcohol is not toxic to E. coli). Also of key importance was the inability of E. coli to grow on benzyl alcohol. Similarly, it was important to demonstrate growth of P. putida on benzyl alcohol. Although P. putida is well known for its ability to grow on a wide range of aromatic substrates (Wackett, & Hershberger, 2001), growth on benzyl alcohol has not been reported. The ability of each of the two strains to grow under the conditions used in the method is shown in Table 1. The optical density at inoculation was calculated (based upon the optical density on the inocula) as 0.021 (E. coli) and 0.026 (P. putida). The cultures were incubated shaking at 200 rpm and 30° C. The optical density was measured at 600 nm after incubation for 23.5 and 75 hours.

TABLE 1
Growth of E. coli and P. putida on glucose and
benzyl alcohol in batch culture.

| | |
|---|---|
| | Organism |
| | (Optical density at 600 nm |
| | after 23.5 and 75 hours |
| Carbon source | incubation) |

| | | |
|---|---|---|
| Benzyl | E. coli | P. putida |

| Glucose | alcohol | 23.5 | 75 | 23.5 | 75 |
|---|---|---|---|---|---|
| (0.1 g 1⁻¹) | (1.0 g 1⁻¹) | hours | hours | hours | hours |
| Not added | Not added | 0.026 | 0.027 | 0.050 | 0.046 |
| + | Not added | 0.116 | 0.102 | 0.170 | 0.138 |
| + | + | 0.102 | 0.097 | 0.042 | 0.353 |
| Not added | + | 0.027 | 0.024 | 0.021 | 0.417 |

From the data shown in Table 1 it is clear that E. coli can grow on glucose in DM and cannot grow on benzyl alcohol in DM, but will grow in the presence of 1.0 g l⁻¹ benzyl alcohol. It is important that E. coli is able to tolerate benzyl alcohol as this will be present in the feed fluid for the entire experiment. It was establised that P. putida was able to grow in DM containing both glucose and benzyl alcohol.

2.2 Measurement of a Characterised Population Shift Using the Method of the Invention

DM was inoculated with E. coli to give a starting optical density (measured at 600 nm) of 0.06 and then operated in batch mode for 19 hours during which time the BOD increased to approximately 200 mg l⁻¹. The BOD then declined rapidly indicating that the glucose in the medium was exhausted. When the fresh medium was pumped into the vessel the BOD increased again, peaking at just over 200 mg l⁻¹ before stabilising at ˜185 mg l⁻¹. Based on the calculated BOD for a feed fluid containing 0.5 g l⁻¹ glucose the BOD was expected to be 178 mg l⁻¹ (see calculation below).

Balance the stoichiometry of the following equation:
C₆H₁₂O₆+O₂.CO₂+H₂O
i.e.
C₆H₁₂O₆+6O₂.6CO₂+6H₂O

Therefore:

Complete oxidation of 1 mol of C₆H₂O₆ requires 6 mol of O₂

Convert from moles to grams:

180.2 grams of C₆H₁₂O₆ requires 32×6 grams O₂

180.2 g of C₆H₁₂O₆ requires 192 g of O₂

Concentration of glucose in the feed=0.5 g l⁻¹, therefore:

0.5 g of C₆H₁₂O₆ requires 0.53 g of O₂

Therefore the Chemical Oxygen Demand (COD):
COD=530 mg l⁻¹

The BOD is assumed to be one third of the COD:
BOD=178 mg l⁻¹

The correction factor for conversion of COD to BOD was determined experimentally using acetate as the carbon source. The BOD of a known concentration of acetate was determined experimentally and compared to the calculated COD for the same concentration of acetate and the difference was found to be three-fold. It is assumed that the same conversion factor can be used for a range of readily biodegradable substrates.

The actual BOD was slightly higher than the calculated BOD for the substrate due to background respiration of the culture. Background respiration can be attributed to maintenance energy production and is therefore dependent upon the biomass concentration in the reactor. As the substrate concentration was relatively low the biomass concentration was also low and similarly the background respiration was low. Background respiration can be determined after the culture has reached steady state. The feed fluid flow is reduced to 0 ml h⁻¹ and a rapid decrease in BOD is observed. Despite the absence of any readily degradable carbon the BOD is usually greater than zero. After a period of stabilisation the BOD will attain a steady value which is an indication of the background respiration.

Based on the BOD it was clear that steady state had been attained (it is generally assumed that steady state has been established after turnover of at least three vessel volumes which, in this example would occur after 37.5 hours). After 125.7 hours (equivalent to 10 vessel volumes) of continuous operation, P. putida was added to the culture. Initially, there was no change in the BOD so to ensure P. putida was not being washed out of the vessel the feed flow rate was reduced from 60 ml h⁻¹ to 30 ml h⁻¹. The BOD increased slowly indicating that degradation of benzyl alcohol was beginning to occur. This observation was confirmed by measuring the residual benzyl alcohol in the culture supernatant, which had started to decrease. As the P. putida population developed the BOD increased, peaking initially at nearly 1400 mg l⁻¹ before declining to 1050 mg l⁻¹ after which a second peak in the BOD was observed. The reason for the oscillation in the BOD is not clear although before reaching steady state microbial populations can demonstrate oscillations as the system equilibrates. After the second BOD peak, the BOD stabilised at 1040 mg l⁻¹ which was the expected BOD for a feed containing 0.5 g l⁻¹ glucose and 1.0 g l⁻¹ benzyl alcohol (see calculation below).

Balance the stoichiometry of following equation:
C₇H₈O+O₂.CO₂+H₂O
i.e.
2C₇H₈O+17O₂.14CO₂+8H₂O

Therefore:

Complete oxidation of 2 mol of C₇H₈O requires 17 mol of O₂

Convert from moles to grams:

108.1×2 grams of C₇H₈O requires 32×17 grams O₂

216.2 g of C₇H₈O requires 544 g of O₂

Concentration of benzyl alcohol in the feed=1.0 g l⁻¹, therefore:

1.0 g of C7H8O requires 2.52 g of O₂

Therefore the Chemical Oxygen Demand (COD):
COD=2516 mg l⁻¹

The BOD is assumed to be one third of the COD:
BOD=839 mg l⁻¹

As the feed contains both 0.5 g l⁻¹ glucose and 1.0 g l⁻¹ benzyl alcohol expected output when both substrate are being used by the microbial population in the reactor:
BOD=839+178=1017 mg l⁻¹

The feed consisting of DM containing 0.5 g l⁻¹ glucose and 1.0 g l⁻¹ benzyl alcohol was fed into a vessel at 30° C. and pH 7. The feed flow rate was initially 60 ml h⁻¹. As shown in FIG. 6, after inoculation of the reactor with E. coli (Arrow A), a population of microorganisms which could only use glucose as a carbon source for growth was established (Arrow B). P. putida, which can use benzyl alcohol as a carbon source for growth, was then added to the reactor (Arrow C) and the feed flow rate was reduced to 30 ml h⁻¹ (Arrow D). A resultant increase in BOD and decrease in benzyl alcohol concentration were observed (Arrow E). The residual benzyl alcohol concentration was estimated using gas chromatography. At the same time that the BOD stabilised the measured residual benzyl alcohol concentration was zero. Interestingly, with a feed flow rate of 30 ml h⁻¹ steady state was expected to be attained after 75 hours. However, based on the BOD, steady state that was not achieved until 94 hours after the feed flow rate was reduced from 60 ml h⁻¹ to 30 ml h⁻¹. From this observation the microbial discovery process will be improved by waiting at least four vessel volumes before assuming a microbial population has reached steady state.

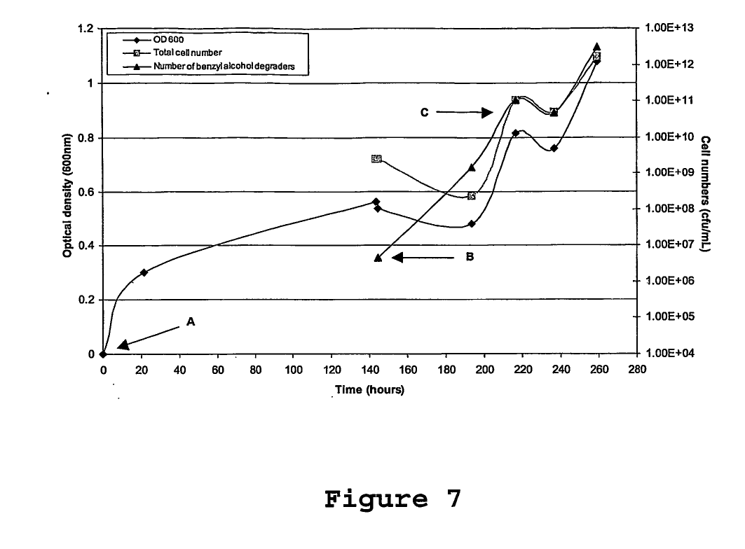

During the course of the experiment the biomass concentration was also monitored as was the number of benzyl alcohol-degrading microorganisms in the population (FIG. 7). Viable cell numbers were estimated by plating samples of the culture (diluted in DM with no added carbon source) on to solid DM containing either 1.0 g l⁻¹ glucose or 1.0 g l⁻¹ benzyl alcohol. The optical density of the culture was measured at 600 nm; samples were diluted in water if the optical density was greater than 0.4. After inoculation of the reactor with E. coli (Arrow A), a population of microorganisms which could only use glucose as a carbon source for growth was established. P. putida, which can use benzyl alcohol as a carbon source for growth, was then added to the reactor (Arrow B). A resultant increase in optical density, the total number of viable cells and the number of cells that could grow on benzyl alcohol, was observed (Arrow C). The observed increase in biomass-concentration (FIG. 7) correlated with the increase in BOD shown in FIG. 6. The E. coli population growing on glucose and at steady state contained 2.52×10⁹ cfu (colony forming units) ml⁻¹ of culture, none of which could grow on benzyl alcohol. The inability of the E. coli population to grow on benzyl alcohol was confirmed by plating undiluted culture onto defined medium with benzyl alcohol as the only carbon source. Immediately after addition of P. putida to the culture the number of microorganisms growing on benzyl alcohol increased to 4.47×10⁶ cfu ml⁻¹. In parallel with the increase in the BOD, the number of microorganisms in the population capable of degrading benzyl alcohol increased. As expected, the total number of benzyl alcohol-degrading microorganisms and the optical density of the culture increased as the BOD increased and the benzyl alcohol concentration decreased. When the population approached steady state the number of benzyl alcohol-degrading microorganisms had increased to greater than 10¹² cfu ml⁻¹ (FIG. 7), an observation clearly reflected in the BOD. These data demonstrate the utility of BOD for on-line real-time monitoring of the status of a microbial discovery process. Unlike the BOD, both analysis of residual benzyl alcohol concentration by gas chromatography and off-line measurement of biomass concentration take time and do not provide an instant assessment of the activity of a microbial population.

Using an experimental procedure which enabled the development of a microbial culture growing exclusively on glucose and the introduction of a population change that could be readily characterised, on-line BOD and off-line data from conventional analytical techniques were compared. Comparison of the BOD with off-line analyses such as optical density, viable count and residual substrate concentration clearly demonstrated that the changes observed in the off-line data were also observed in the BOD. The inverse relationship between BOD and residual substrate concentration that was demonstrated in batch culture was also observed in the continuous system. These data demonstrate the utility of BOD for use in real-time monitoring of the effect of changes to a microbial population growing in continuous culture.

### Example 3 Use of BOD for Microbial Discovery

Discovery of 1-methyl-2-pyrrolidinone-Utilising Microorganisms

Discovery of 1-methyl-2-pyrrolidinone-utilising microorganisms was performed using the method of the invention by imposing selective pressure (in this case the ability to utilise 1-methyl-2-pyrrolidinone as a sole source of organic carbon and energy) in unison with BOD. The method was performed in the apparatus of FIGS. 1 and 2. A population of microorganisms with the required characteristics was readily established.

Fresh activated sludge sourced from a wastewater treatment facility was used as the source of microorganisms for enrichment of 1-methyl-2-pyrrolidinone-utilising microbes. As 1-methyl-2-pyrrolidinone is soluble in water it was added to the feed fluid at the 1 g/l concentration. The enrichment process was performed at 30° C. and pH 7.0 (the pH was maintained at 7.0 by the automatic addition of a potassium hydroxide or hydrochloric acid solution as the alkali and acid, respectively). The feed flow rate was 60 ml h⁻¹.

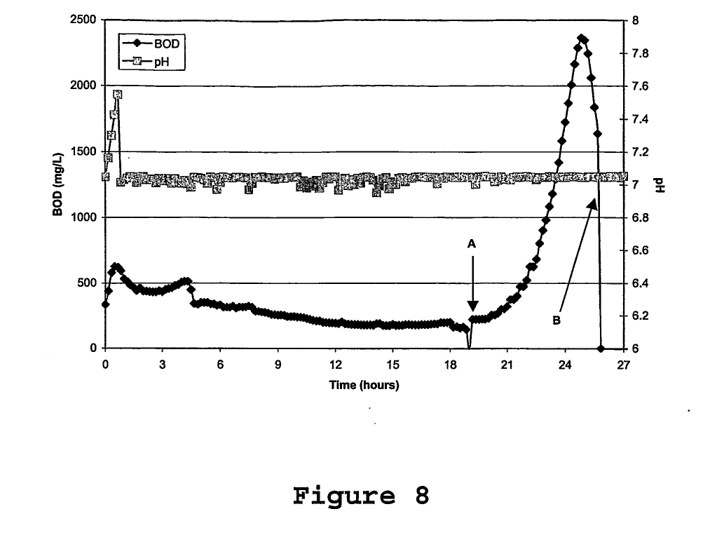

After the addition of the activated sludge to a vessel the BOD was high (greater than 500 mg l⁻¹). The activated sludge had a high initial BOD because it contained residual readily biodegradable carbon which was gradually degraded, resulting in the observed gradual decline in BOD before the addition of 1-methyl-2-pyrrolidinone. After 2 ml of 1-methyl-2-pyrrolidinone was added to the vessel (Arrow A; FIG. 8) a rapid rise in BOD was observed indicating exponential growth (FIG. 8). This data can be used to calculate μmax (maximum doubling time) for the population that is growing on the substrate. Growth is exponential between 1220 and 1460 minutes and μmax can therefore be calculated as 0.52 h⁻¹, which corresponds to a doubling time of 1.34 hours. The rapid decline in BOD (Arrow B, FIG. 8) was due to the oxygen consumption of the microbial population being greater than the amount of oxygen supplied to the culture.

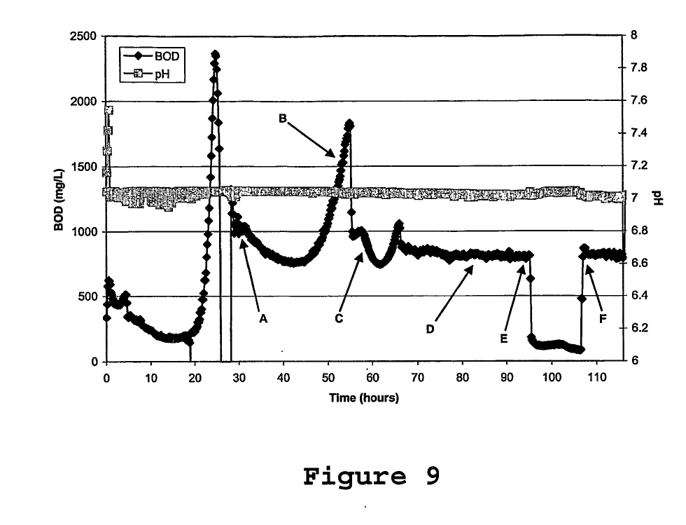

After initial batch operation, the system was operated in continuous mode (FIG. 9) and the feed fluid was increased to a flow rate of 60 ml h⁻¹. It should be noted that when the feed was started the BOD appears to be very low. This is not a true reflection of the status of the culture; the BOD was in fact off-scale (too high) and could not be measured accurately. After the feed pump was started a second exponential rise in BOD was observed which could be attributed to unbalanced growth. The culture will take a period of time to adjust to the fluid flow rate with the usual result being a build-up of the limiting nutrient that is then rapidly depleted as all nutrients are once again in excess. The rapid decrease in BOD after the exponential rise signifies depletion of excess 1-methyl-2-pyrrolidinone as the system approaches equilibrium. The BOD then stabilised at approximately 800 mg l⁻¹, which is the expected value from a feed containing 1.0 g l⁻¹ 1-methyl-2-pyrrolidinone (see calculation below):

Balance the stoichiometry of the following equation:
C₅H₉₀N+O₂.CO₂+H₂O+NH₃
i.e.
4C₅H₉₀N+27O₂.20CO₂+12H₂O+4NH₃

Therefore:

Complete oxidation of 4 mol of C₅H₉NO₂ requires 27 mol of O₂

Convert from moles to grams:

99.13×4 grams of C₅H₉NO₂ requires 32×27 grams O₂

396.5 g of C₅H₉NO₂ requires 864 g of O₂

Concentration of 1-methyl-2-pyrrolidinone in the feed=1 g l⁻¹, therefore:

1 g of C H NO requires 2.18 g of O₂

Therefore the Chemical Oxygen Demand (COD):
COD=2180 mg l⁻¹

The BOD is assumed to be one third of the COD:
BOD=726 mg l⁻¹

The calculated BOD for 1 g l⁻¹ 1-methyl-2-pyrrolidinone is less than the measured BOD output. Once again, the difference is probably due to background respiration. As expected, when the flow of the feed fluid was reduced to 0 ml h⁻¹ the BOD dropped rapidly and remained constant at approximately 80 to 120 mg l⁻¹. This background respiration needs to be subtracted from the measured BOD output to give a true indication of the BOD and therefore the measured and calculated BOD are approximately the same. The absolute BOD is not critical for the success of the method of the invention. For microbial discovery the relative value gives a better reflection of the status of a discovery process. For example, the large peak in BOD at the start of the experiment (FIG. 9) gives a clear indication of microbial attack of the substrate. The calculated BOD can be used as a guide to select substrate concentrations and other operating parameters. For example by calculating the BOD of a particular substrate the operator can ensure that the substrate concentration in the feed does not exceed the measurable BOD output.

After 116 hours the feed flow rate was increased to 120 ml h⁻¹ and shortly after the 1-methyl-2-pyrrolidinone concentration in the feed was increased to 2 g l⁻¹ (data not shown). This was continued for a further 95 hours after which a sample was taken for isolation of pure cultures of, the microorganisms that were present in the culture. The sample was heavily aggregated with large flocs present and microscopic examination revealed a culture that was dominated by a non-motile rod with a low number of motile rods also being present. The sample was plated onto solid defined medium with 1-methyl-2-pyrrolidinone as the sole carbon source and the plates incubated at 30° C. for ˜40 hours. From these plates three isolates, designated 2A, 2B and 2C, were purified. Based on microscopic appearance and colonial morphology 2A and 2C were assumed to be the same organism and 2C was pursued no further.

The characteristics of the pure isolates are shown in table 2:

TABLE 2
Colony morphology and microscopic characteristics
of the 1-methyl-2-pyrrolidinone-degrading isolates
designated 2A and 2B.

| Isolate 2A | | Isolate 2B | |
|---|---|---|---|
| Colonial | Microscopic | Colonial | Microscopic |
| morphology | appearance | morphology | appearance |
| Mucoid | Rod shape | Slightly | Slightly bent, |
| | | mucoid | possibly cocci |
| | | | in chains |
| Semi opaque | Slight | Yellow/white | Non motile |
| | motility | | |
| Off white/grey | Short rods | 1 mm diameter | Cocco-bacilli |
| colour | | | |
| 2-4 mm | Gram negative | Round colonies | Gram positive |
| diameter | | | |
| Slimy | | Apparent | |
| | | fluorescence | |
| Apparent | | | |
| fluorescence | | | |

The ability of the pure isolates to grow on 1-methyl-2-pyrrolidinone as the sole source of carbon in liquid culture was also evaluated (Table 3). The cultures were grown in 50 ml screw-capped plastic tubes that contained 10 ml of defined medium and 1.0 g l⁻¹ 1-methyl-2-pyrrolidinone. To ensure each culture was inoculated with a consistent number of cells, 10 ml of medium was seeded with 100 μl of a single colony that had been resuspended in 1 ml of DM. The cultures were incubated at 30° C. shaking at 190 rpm. A single 10 ml culture was harvested by centrifugation at each time point and the supernatant kept for determination of the 1-methyl-2-pyrrolidinone concentration. The 1-methyl-2-pyrrolidinone concentrations were estimated using gas chromatography

TABLE 3
1-methyl-2-pyrrolidinone degradation by isolates
2A and 2B.

| | Residual 1-methyl-2-pyrrolidinone | | |
|---|---|---|---|
| Time after | concentration (mg l⁻¹) | | |
| inoculation | Uninoculated | | |
| (h) | Control | Isolate 2A | Isolate 2B |
| 24 | 1000 | NDa | ND |
| 48 | 960 | ND | ND |
| 72 | 850 | ND | ND |
| 96 | 870 | ND | ND |
| 168 | 900 | ND | ND |

aNot detected (limit of detection · 20 mg l⁻¹).

The results show that from a large mixed population (activated sludge) two isolates were obtained that were able to use 1-methyl-2-pyrrolidinone as the sole source of carbon. Both these isolates were able to completely degrade 1.0 g l⁻¹ 1-methyl-2-pyrrolidinone in batch culture within 24 hours.

The BOD output demonstrates the usefulness of BOD as a real-time monitor of the status of a culture. Any changes to the operating conditions are reflected almost immediately in the visual output. This enables the operator to make changes and note the response of the culture rapidly without the requirement for off-line analyses which are time consuming and result in a delay before the effect of a change can be assessed. Additionally, growth on 1-methyl-2-pyrrolidinone was demonstrated without the need for development of an assay for the substrate. This has the added benefit in that insoluble substrates (see Example 4) can be assessed which can be difficult to assay because a representative sample cannot be taken and analysed easily.

### Example 4 Discovery of Dodecane-Utilising Microorganisms

Discovery of dodecane-utilising microorganisms was performed using the method of the invention. By imposing selective pressure (in this case the ability to utilise dodecane as a sole source of carbon and energy) in unison with monitoring the BOD output, a microbial population with the required characteristics was readily established. As dodecane is practically insoluble in water it was fed into the culture using a separate peristaltic pump at a flow rate of 0.79 ml h⁻¹. The purpose of this example was to discover microbes that could potentially hydroxylate linear hydrocarbons. This is extremely difficult to achieve using convention chemical (non-microbial) techniques.

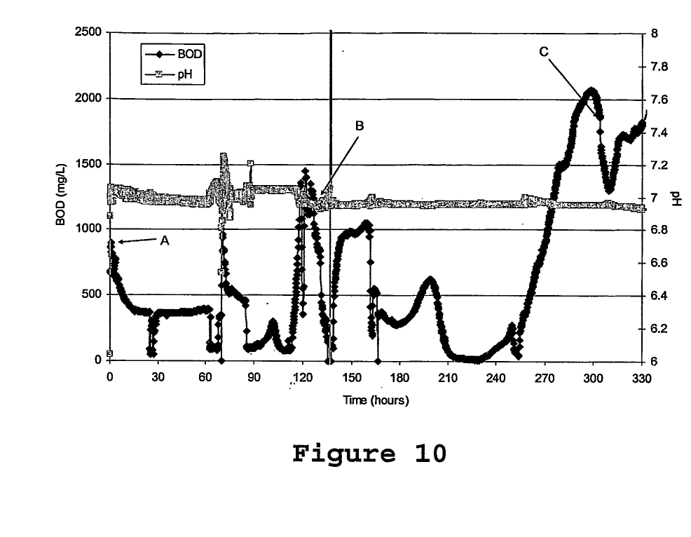

Fresh activated sludge sourced from a wastewater treatment facility was used as source of microorganisms for discovery of dodecane-utilising microbes. The process was conducted on the apparatus of FIGS. 1 and 2. The discovery process was performed at 30° C. and pH 7.0 (the pH was maintained at 7.0 by the automatic addition of a potassium hydroxide or hydrochloric acid solution). The feed was comprised of DM that had no carbon source added and the feed flow rate was initially 30 ml h⁻¹ and the flow of dodecane was 0.79 ml h⁻¹. The experiment was conducted over 137 hours then the apparatus components cleaned and restarted (using the same culture) (Arrow B) with a feed flow rate of 60 ml h⁻¹ (the dodecane flow rate was unchanged). After 330 hours of operation the fluid was sampled to enable isolation of dodecane-degrading microorganisms (Arrow C, FIG. 10).

Although the BOD output was variable, clearly a population of dodecane-degrading microorganisms had been established. The population took somewhat longer to establish than was observed with the water soluble substrate 1-methyl-2-pyrrolidinone. Two possible explanations for this observation are (i) the variability of substrate flow resulted in gradual washout of any dodecane-degrading population that may have become established and/or (ii) the insoluble nature of the substrate reduces microbial attack resulting in slower growth. A combination of gradual washout and an insoluble substrate could result in reduced degradation of the substrate because the enriched population may be producing surfactants or similar molecules that assist in solubilising the substrate. Gradual washout would continually reduce the concentration of any surfactant-type molecules further decreasing the accessibility of the substrate resulting in a continual compounding negative effect. In this experiment dodecane was fed into the reactor using a peristaltic pump which resulted in the variable BOD output. Syringe pumps or peristaltic pumps can be used to feed insoluble substrates into the culture, however a syringe pump is preferred because the product contact components of a syringe pump are compatible with a wide range of chemicals.

The BOD output is significantly less than the calculated value based on the COD of dodecane (see calculation below).

Balance the stoichiometry of the following equation:
C₁₂H₂₆+O₂.CO₂+H₂O
i.e.
2C₁₂H₂₆+37O₂.24CO₂+26H₂O

Therefore, complete oxidation of 2 mol of C₁₂H₂₆ requires 37 mol of O₂

Convert from moles to grams:

170.3×2 grams of C₁₂H₂₆ requires 32×37 grams O₂

340.6 g of C₁₂H₂₆ requires 1184 g of O₂

Assuming the concentration of dodecane in the feed=1 g l⁻¹, therefore:

1 g of C₁₂H₂₆ requires 3.48 g of O₂

Therefore the Chemical Oxygen Demand (COD):
COD=3480 mg l⁻¹

The BOD is assumed to be one third of the COD:
BOD=1158 mg l⁻¹

Actual flow rate of dodecane=0.788 ml h⁻¹=0.591 g h⁻¹

Therefore the estimated dodecane concentration in feed=0.591/60 ml=9.85 g l⁻¹

Expected COD=28861 mg l⁻¹ and BOD=9620 mg l⁻¹.

This result was unexpected although it could be explained by the insolubility of the substrate. As dodecane is less dense than water it will tend to float to the surface of the culture, particularly during the measurement of oxygen uptake where aeration has stopped and the stirrer has slowed. Much of the dodecane may be washed out in the overflow. The measured BOD may also be an indication of the amount of substrate that is accessible to the microbial population and that is limited by the solubility of dodecane in water.

After 207 hours of growth on dodecane a sample was taken from the enriched culture for isolation of pure cultures. Microscopic examination of the sample revealed a range of rod-shaped bacteria both short and filamentous. Cocci-shaped bacteria were also evident and a number of motile rods were also observed. The sample was plated onto solid DM with dodecane as the sole carbon source and the plates were incubated at 30° C. for ˜48 hours. From these plates four isolates, designated 1A, 1B, 1C and 1D, were purified. Based on microscopic appearance and colonial morphology 1A and 1D were assumed to be the same organism and 1A was pursued no further.

The characteristics of the pure isolates are shown in table 4:

TABLE 4
Colony morphology and microscopic
characteristics of the dodecane-degrading isolates
designated 1B, 1C and 1D.

| Isolate 1B | | Isolate 1C | | Isolate 1D | |
|---|---|---|---|---|---|
| Colonial | Microscopic | Colonial | Microscopic | Colonial | Microscopic |
| morphology | appearance | morphology | appearance | morphology | appearance |
| Shiny | Cocco- | Fried egg | Long and | Small uneven | Long rods |
| | bacilli | appearance | short rods | colonies | |
| Round | Non-motile | Target shaped | Motile | Crinkly | Non-motile |
| | | | | appearance | |
| Off white | Gram | 3-6 mm | Gram | Off white | Gram negative |
| colour | negative | diameter | negative | colour | |
| ˜1.5 mm | | Apparent | | Hazy/opaque | |
| diameter | | fluorescent | | appearance | |
| | | halo | | | |

The ability of the pure isolates to grow on dodecane as the sole source of carbon in liquid culture was also evaluated and is shown in Table 5. The cultures were grown in 50 ml screw-capped plastic tubes that contained 10 ml of defined medium and 0.75 g l⁻¹ dodecane. To ensure each culture was inoculated with a consistent number of cells, 10 ml of medium was inoculated with 100 μl of a single colony that had been resuspended in 1 ml of DM. The cultures were incubated at 30° C. shaking at 190 rpm. Residual dodecane was extracted by the addition of 20 ml of hexane to a single 10 ml culture at each time point. The tube was shaken vigorously for one minute and after phase separation, the upper layer was kept for determination of the dodecane concentration. The dodecane concentrations were estimated using gas chromatography.

TABLE 5
Dodecane degradation by isolates 1B, 1C and 1D.

| | |
|---|---|
| Time after | Residual dodecane concentration (mg l⁻¹) |

| inoculation | Uninoculated | Isolate | Isolate | Isolate |
|---|---|---|---|---|
| (h) | Control | 1B | 1C | 1D |
| 24 | 820 | 920 | 630 | 1080 |
| 48 | 800 | 160 | 620 | 920 |
| 72 | 200 | 420 | 440 | 740 |
| 96 | 740 | 270 | 440 | 410 |
| 168 | 740 | 290 | 280 | 310 |

The results show that from a large mixed population (activated sludge) three isolates were obtained that were able to use dodecane as the sole source of carbon. In batch culture the isolates were able to use (over a 168 hours period) 50 to 60% of the dodecane added to the culture. The rate of dodecane utilisation is substantially slower than 1-methyl-2-pyrrolidinone which may be due to the difference in the solubility of the two compounds (dodecane is practically insoluble in water). The insolubility of dodecane may impose mass transfer limitations which will slow growth and utilisation of the substrate significantly. The variability in the gas chromatography data from the batch experiments highlights the difficulties associated with analysis of concentrations of insoluble substrates. This problem can be partly overcome by monitoring BOD, as oxygen consumption can be used as an indirect indicator of growth on the substrate.

### Example 5 Discovery of Olive Oil-Utilising Microorganisms

The use of olive oil as a feed fluid highlights another of the advantages of the method of the invention, which is microbial discovery in extreme environments. Olive oil is a heterogeneous substrate of which development of an analytical method for measuring consumption would be difficult. Monitoring BOD enables demonstration of growth on this complex substrate without the requirement for the development of complex analytical methods. The isolation of microorganisms capable of using substrates such as olive oil for growth may enable the discovery of lipases with useful properties. The following experiment was performed to facilitate not only the isolation of olive oil-degrading microorganisms but also to enrich microbes that can tolerate a very broad pH range.

The vessel was filled with activated sludge and 10 ml of olive oil was added. The BOD rose rapidly and peaked at ˜1700 mg l⁻¹. The rapid onset in the ability to degrade olive oil in a population of microorganisms from activated sludge is not unexpected as the presence of this type of substrate in the influent streams of wastewater treatment facilities is highly likely. After the peak in BOD was observed (20.5 hours) olive oil was fed continuously into the vessel as was a separate stream from DM mixed with activated sludge in the ratio 4:1. The pH set point was reduced to pH 4.0 and after 225 hours the feed medium was changed from a mixture of DM and activated sludge to DM. No changes were made to the conditions for 138 hours (equivalent to 11 vessel volumes) and the BOD of the culture remained high. From these observations it was concluded that a microbial population had been established that could use olive oil as the sole source of carbon at pH 4.0.

The olive oil flow rate was reduced to 0.061 ml h⁻¹ and the feed was again changed to a mixture of activated sludge and DM. These conditions resulted in a culture with activity (BOD in the range 1200 to 1500 mg l⁻¹) then after 458 hours the pH set point was changed to pH 2.2, the feed was again changed to DM without any additions and the feed flow rate was increased to 66 ml h⁻¹. The conditions were unchanged for 55 hours (4.4 vessel volumes) and the BOD stabilised at ˜1700 mg l⁻¹ indicating that a population of microorganisms had been established that was capable of using olive oil as a sole source of carbon at pH 2.2.

The next phase of the experiment evaluated the ability of the microbial population that had been growing at pH 2.2 to respond to an increase in the pH of the culture. At 555 hours the pH set point was increased to 9.0. A further increase in the pH set point from pH 9.5 to 10 resulted in another decline in BOD indicating washout and/or death of the microbial population. Interestingly, when the pH was reduced by just 0.5 of a pH unit the culture recovered with the BOD increasing exponentially. The culture showed significant sensitivity to pH values greater than 9.5. The reason for this observation is not clear however two possible explanations for the increased sensitivity to pH 10 could be (i) one of the medium components was insoluble at pH 10 resulting in significant nutrient limitation and a decline in BOD or (ii) the microbial population present in the culture had not adapted to growth at pH 10. The culture was maintained at pH 9.5 for 125 hours and clearly a population of microorganisms growing on olive oil as the sole carbon source at pH 9.5 had been established. It cannot be concluded that this population also has the potential to grow at pH 2 because the time taken to establish this population may have resulted in the development of a totally new population that is better adapted to growth under the new conditions.

Over a 37 day period growth of a microbial population on olive oil at a range of pH values was demonstrated. The two extremes of pH were 2.2 and 9.5. Clearly microbial activity could be demonstrated at these pH values and these data were then used to develop an automatic pH oscillation system; this system was designed to facilitate the isolation of microorganisms with tolerance to a broad pH range with the view to isolating enzymes from these microorganisms which exhibit similar pH tolerance (both activity and stability).

### Example 6 Development of a Feedback Loop Between Feed Flow Rate and OUR

A feedback loop between feed flow rate and OUR was developed to enable the maximum growth rate of a microbial population to be established using an automated system. The maximum growth rate of a population is an important parameter as this is likely to give an indication of the rate of flux through a metabolic pathway and therefore an indication of the activity of enzymes in the pathway.

6.1 Design of Feedback Loop

The feedback loop uses the limiter that if the BOD remains within a set range for an operator set period then the flow rate is increased by a value that is also specified by the operator. This is described briefly above in relation to the apparatus of the embodiment illustrated in FIGS. 1 and 2. The software to run the feedback loop was developed using a commercially-available software package used to write control software.

6.2 Testing of the Feedback Loop Between Feed Flow Rate and OUR

Isolation of 1,3-propanediol-degrading microorganisms was used to test the feedback loop between feed flow rate and OUR. The feed medium was a defined medium designated 461S (Appendix I) which contained 1.0 g l⁻¹ 1,3-propanediol and the initial flow rate was 43.5 ml h⁻¹. The operating temperature was 30° C. and pH 7.0. The medium was inoculated with ˜700 ml of activated sludge. The feed flow rate was increased by 20 ml h⁻¹ if the BOD remained constant for four vessel volumes. After an initial peak in the BOD, which was due to 1,3-propanediol being in excess, the BOD remained constant over a range of flow rates. The flow rate was increased in steps from 43.5 ml h⁻¹ to 143.5 ml h⁻¹ over several days without any significant change in the BOD, demonstrating that the microbial population which had been established was capable of growing with doubling times in the range 3.6 to 12 hours. At a doubling time of 3.6 hours the BOD was unchanged suggesting that the population that had been established had the ability to grow faster than the maximum that was tested in this experiment. This observation was expected because the inferred μmax of the population from the initial peak in BOD at the start of the experiment was ˜0.25 h⁻¹ (a doubling time of 2.8 hours). Higher doubling times could be achieved with any microbial population that is established in the culture because there is a good probability that mutants which can grow at a higher rate will be selected at high feed flow rates.

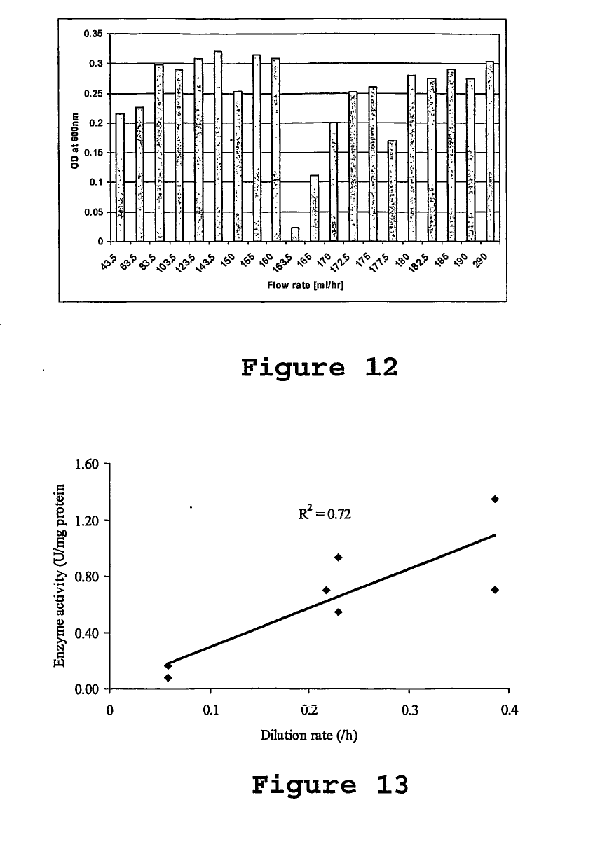

The response of the culture to the changing flow rates is shown in FIG. 12.

### Example 7 Use of Method in Enzyme Discovery

The aim of this example was to demonstrate that the method can be used to discover specific enzymes and that the kinetic behaviour of the enzymes could be selected and controlled. The method was used to demonstrate (i) the discovery of 1,3-propanediol dehydrogenase activity and (ii) the specific activity of the discovered enzyme could be altered in a controlled way during the course of the discovery process. For this purpose, 1,3-propanediol was used as the sole carbon source. It was assumed that in prokaryotic systems, oxidoreductases are amongst the first class of enzymes used to degrade a carbon source therefore the likelihood of discovering a dehydrogenase specific for 1,3-propanediol was high. Furthermore, by utilising the feedback loop as described in Example 6, it was anticipated that an increase in the feed flow (i.e. dilution rate) would result in selection of microbes that had high 1,3-propanediol dehydrogenase activity; increased dehydrogenase activity enabling faster metabolism of the 1,3-propanediol. [i.e. Microorganisms with a higher enzyme activity would be expected to proliferate at higher dilution rates (high feed flows)]. If this assumption is correct then it would be indicated, at a cursory level, by an increase in the specific activity of 1,3-propanediol dehydrogenase in microbial isolates recovered from the method at high dilution rates.

The feed medium was the defined medium 416S set out in the second part of Appendix 1 which contained 1.0 g l⁻¹ 1,3-propanediol. The operating temperature was 30° C. and pH 7.0. The reactor was inoculated with ˜700 ml of mixed microbial population (activated sludge) suspended in water. Dilution rates ranged from 0.058 h⁻¹ to 0.387 h⁻¹.

To determine the biomass concentration in the system, samples were taken after each flow rate change and after a minimum of three vessel volumes had passed through the system. Optical density measured at 600 nm was used as a measure of the biomass concentration. Samples were taken after the culture had reached steady state and before the flow rate increased. Optical density stabilised at approximately 0.3 after each flow rate change but dropped significantly at a flow rate of 163.5 ml h¹ (FIG. 12). The decrease in biomass concentration correlated with a decrease in the diversity of the microbial population in the vessel. Further increases in the flow rate did not result in washout of the culture, in fact the optical density of the culture recovered and continued to be maintained at ˜0.3 up to a flow rate of 290 ml h⁻¹.

The samples taken at each dilution rate were plated onto 416S medium with 1,3-propanediol added and colonies with different morphology were established as pure cultures. A total of 66 isolates were obtained. Identification of one of the selected isolates using was undertaken using 16S ribosomal RNA sequencing. Isolate number 7#1 showed 98% homology with the 16S ribosomal RNA sequence of Gordonia desulfuricans. Although the production of 1,3-propanediol has been shown in several other bacterial genera including Klebsiella, Enterobacter, Citrobacter, Lactobacillus and Clostridium (Huang 2002, Nakamura 2003), to our knowledge Gordonia species have never before been reported to be associated with 1,3-propanediol metabolism again highlighting the utility of the method for the isolation of unique microorganisms, or the discovery of new activities for microorganisms.

Seven isolates obtained at a range of flow rates were chosen for further study. The activity of 1,3-propanediol dehydrogenase was measured in cell-free extracts obtained from each of the isolates after growth in batch culture with 1,3-propanediol as the carbon source (Table 6).

TABLE 6

| | | Specific activity |
|---|---|---|
| Isolate number | Flow rate (ml h⁻¹) | (U mg protein⁻¹) |
| 1#3 | 43.5 | 0.078 |
| 1#4 | 43.5 | 0.164 |
| 9#5 | 163.5 | 0.696 |
| 16#1 | 172.5 | 0.543 |
| 16#4 | 172.5 | 0.935 |
| 24#1 | 290 | 0.701 |
| 24#2 | 290 | 1.347 |

Enzyme activity was detected in cell-free extracts. Cell-free extracts were obtained after harvesting shake flask cultures during early stationary growth phase by centrifugation at 12227×g, 4° C. for 15 minutes. Cell pellets were washed in 50 mM HEPES-buffer containing 100 μM MnCl₂. The cell pellets were then resuspended in a volume of 50 mM Tris-HCl pH 8.0 that contained 1 mM ETDA, 0.1% Triton X-100, 1 mM PMSF, 2 mM MgCl₂, 0.5 mg ml⁻¹ lysozyme, 5 μg ml⁻¹ DNAse equivalent to the pellet weight. The cells were lysed by adding 1 gram of glass beads per ml of suspended cells and vortexing for 1 minute. The lysate was separated from the glass beads and cell debris by centrifugation at 12000×g, 4° C. for 5 minutes. Enzyme activity was determined in quartz cuvettes by measuring the formation of NADH at 340 nm over a period of one minute. The reaction mixture consisted of 0.05 M Na2CO3 (pH 9.5), 2 mM NAD+, 0.1 M 1,3-propanediol and 50 μl cell free extract in a final volume of 1 ml. All enzyme assays were performed in triplicate and averaged. One unit of enzyme activity is equivalent to the formation of one micromole of product per minute. The protein concentration in the cell-free extracts was measured by the method of Bradford (Bradford, 1976) with BSA as the standard. Protein analyses were performed in triplicate.

By increasing the dilution rate we demonstrated a correlation between the substrate flow and the specific activity of 1,3-propanediol dehydrogenase. This is shown in FIG. 13. The enzyme activity test showed that microorganisms isolated at a high dilution rate had an increased specific enzyme activity.

This example therefore demonstrates that the method can be used to specifically discover a chosen enzyme activity. The example also demonstrates that the specific activity of the chosen enzyme can be controlled using dilution rate and the feedback loop and that microbes with previously undescribed phenotypes may be isolated.

### Example 8 Use of the Method for Discovery of Extremophiles

8.1 Use of the Method for Discovery of Psychrophiles

The aim of this experiment was to demonstrate that psychrophilic microorganisms could be isolated from a readily available source of microorganisms using the method. According to Stanier et al. (1987), psychrophilic microorganisms are defined as able to grow well at 0° C. However it should be noted that classification based on temperature is somewhat arbitrary because it does not take into account the temperature range over which growth is possible for a particular isolate. For example Xanthomonas pharmicola can grow at temperatures ranging from 0 to 40° C. and is classified as a psychrophile. For this experiment 4° C. was chosen as the growth temperature to avoid freezing of the growth medium that was expected to occur at lower temperatures. The discovery process could be performed at lower temperatures although operation at temperatures less than 4° C. would require the addition of extra solutes to the medium to prevent freezing.

Discovery of psychrophiles was performed using the method of the invention, on the apparatus described above. By imposing selective pressure (in this case the ability to utilise acetate as a sole source of carbon at 4° C.) a population of microorganisms with the required characteristics was readily established.

A mixed microbial population suspended in water was used as source of microorganisms for discovery of psychrophilic microorganisms. The discovery process was performed at 4° C. and pH 7.0 (the pH was maintained at 7.0 by the automatic addition of ammonium hydroxide or phosphoric acid solutions). To prevent growth in the feed line the test substrate (carbon source) was added to the vessel separately from the nutrients. The nutrient feed was the defined medium 416S of Appendix 1. The nutrient feed flow rate was 20 ml h⁻¹ and the substrate (16.6 g l⁻¹ sodium acetate trihydrate) flow was 6 ml h⁻¹—total 26 ml h⁻¹. These parameters resulted in a doubling time of 18.4 h (corresponding to a dilution rate of 0.038 h⁻¹) and a calculated feed acetate concentration of 2.3 g l⁻¹.

After the addition of the microbial population to the vessel the BOD was high (greater than 600 mg l⁻¹). The gradual decline in BOD was probably due to consumption of any residual readily biodegradable carbon in the activated sludge.

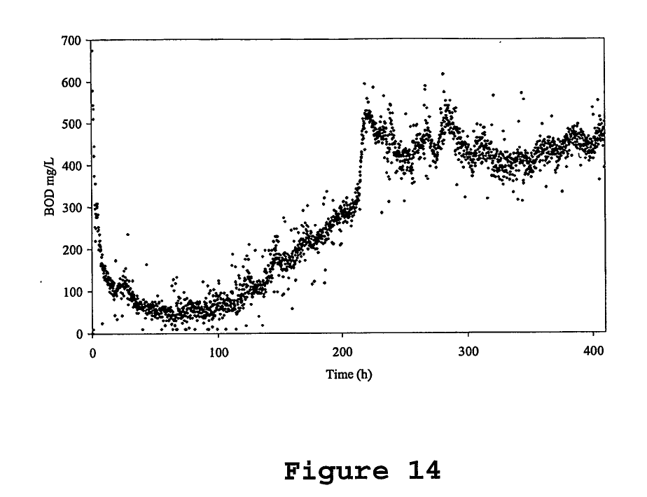

FIG. 14 is a graph of output (in terms of BOD) over time during growth of microorganisms from activated sludge on acetate at 4° C.

The output did not increase markedly for the first 100 hours (˜4 days) of operation after which a gradual increase in BOD was observed. The BOD peaked after ˜220 hours (˜9 days) of operation and stabilised after 300 hours. The time taken for a significant increase in activity of the culture to be observed was far greater than typical operation at 30° C. This highlights the severity of the imposed conditions (low temperature) in the method and the impact of extreme conditions on cellular processes. These observations also highlight the value of the method in providing a real time assessment of the status of a culture, a feature that is important when attempting to find microorganisms that perform a desired function under extreme environmental conditions or transform a particularly recalcitrant compound. The optical density of the culture was periodically measured at 600 nm between 234 hours and 402 hours and averaged at 1.36. The fact the optical density was maintained at ˜1.3 over a period of 168 hours demonstrates that there was a population of psychrophilic microorganisms in the vessel that were able to survive and reproduce at a temperature of 4° C. A constant biomass concentration in the vessel is consistent with the output of the method (which was also constant over the same time period) again demonstrating that the output can be used as a real time indirect measure of the status of a microbial population.

Microscopic examination of the culture showed chains of large rod-shaped bacteria (or small yeast) and small motile bacteria; the presence of fugal hyphae was also noted.

After 266 hours the flow rate of the substrate pump was increased thereby changing the acetate concentration in the feed from 2.3 g l⁻¹ to 3.1 g l⁻¹. The increase in acetate concentration was expected to result in an increase in BOD and optical density however no increase in either parameter was observed. Similarly, a feed containing 2.3 g l⁻¹ acetate was expected to attain a BOD of ˜720 mg l⁻¹ but the output stabilised at 400 to 450 mg l⁻¹. From these observations it can be inferred that either the temperature was limiting growth rather than the carbon source, use of carbon or another nutrient is less efficient at low temperatures, or growth at low temperatures requires excess levels of one or more nutrients other than the carbon source.

By operating the method at 4° C. a psychrophilic microbial population growing on acetate as the carbon source at 4° C. was established. Although the discovery process was slower at 4° C. than is typically observed at 30° C. (an observation that was not unexpected as many cellular processes are likely to slow at lower temperatures), a psychrophilic population was nevertheless established. This example therefore demonstrates that the method is very versatile, and can even can be used to isolate microorganisms that grow at low temperatures.

8.2 Use of the Method for Discovery of Thermophiles

The aim of this experiment was to demonstrate that thermophilic microorganisms could be isolated from a readily available source of microorganisms using the method of the invention. Thermophilic microorganisms are defined as organisms that live at elevated temperatures (Brock and Madigan, 1988). This definition is subjective and can be clarified somewhat with an example of a microorganism that fits the definition. An example of a thermophilic microorganism is Thermus which has an optimum growth temperature of ˜60° C. and can grow at temperatures ranging from 42° C. to 69° C. Extreme thermophiles have also been defined with members of this group being recognised as having very high temperature optima. For example, Thermococcus has an optimum temperature for growth of ˜87° C. (Brock and Madigan, 1988).

Discovery of thermophiles was performed using the apparatus and techniques described above. By imposing selective pressure (in this case the ability to utilise acetate as a sole source of carbon at 80° C.) it was anticipated that a population of thermophilic microorganisms would be established. The measurement of dissolved oxygen concentration at high temperatures can be problematic because the baseline output of a number of dissolved oxygen electrodes is very high at high temperatures. This problem is further compounded by the effect of temperature on the solubility of oxygen. As the temperature of water increases the solubility of oxygen in the water decreases and therefore reliable measurement of dissolved oxygen concentrations at high temperatures is essential. To enable discovery of thermophiles, the apparatus described at the outset of the Examples was modified to enable the installation of a dissolved oxygen electrode that could operate at high temperatures (up to 80° C.). The vessel was also modified to improve its thermal tolerance and the heat input was enhanced with the use of an improved heating system.

A mixed microbial population suspended in water was used as source of microorganisms for discovery of thermophilic microorganisms. The discovery process was performed at 80° C. To prevent growth in the feed line the test substrate-a carbon source (acetate)—was added to the vessel separately from the nutrients. The nutrient feed was the defined medium 461S described in Appendix 1. The nutrient feed flow rate was 52 ml h⁻¹ and the substrate (16.6 g l⁻¹ sodium acetate trihydrate) flow was 1.9 ml h⁻¹. These parameters resulted in a doubling time of 9 h and a calculated feed acetate concentration of 0.26 g l⁻¹.

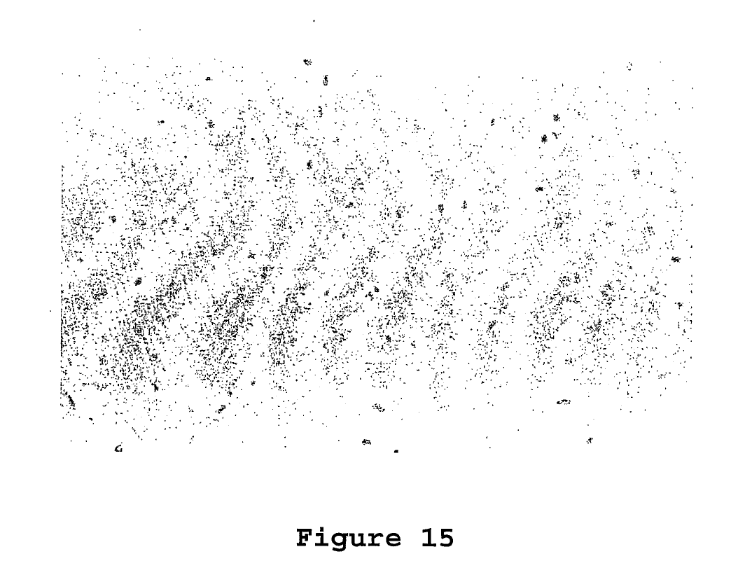

After 16 days no significant microbial activity was detected in the vessel (as measured by oxygen consumption). At 80° C. the measured dissolved oxygen concentration at saturation was ˜3 mg l⁻¹ (c.f. at 30° C. the dissolved oxygen concentration at saturation is ˜7 mg l⁻¹). To demonstrate that the method and apparatus was capable of detecting changes in dissolved oxygen concentration at 80° C. and the dissolved oxygen electrode was operating correctly, the vessel was sparged with nitrogen at the end of an aeration cycle. During nitrogen sparging the output of the dissolved oxygen probe decreased to less than 1 g l⁻¹ indicating that changes in dissolved oxygen could be measured at 80° C. It should be noted that a very small change in dissolved oxygen concentration was observed during this experiment. This change was ˜0.002 mg O₂ per minute. Although this change in dissolved oxygen was probably beyond the sensitivity of the apparatus components, the culture was examined microscopically after 16 days of operation. The micrograph of the sample is set out in FIG. 15.

The examined sample was 1.2 ml in volume, and was centrifuged for 2 minutes and resuspended in ˜25 μl of medium. The concentrated sample was examined as a wet mount by phase contrast microscopy at a magnification of 100×.

The micrograph clearly shows the presence of small rod-shaped bacterial cells—see FIG. 15. The cell numbers were very low (given the sample for the micrograph was concentrated ˜50-fold) which correlated with the very low output of this trial. Although not apparent in the micrograph, some of the rod-shaped cells were motile giving a clear indication that some of the cells were viable. These observations provide evidence to suggest that extreme thermophiles may be recovered using the method of the invention.

Although limited, there is some evidence that suggests that discovery of thermophilic microorganisms may have been facilitated by the method of the invention. Possible reasons for the low activity of the thermophilic microorganisms include (i) acetate is not a preferred substrate of the thermophiles present in the initial population, (ii) the dilution rate was too high for the thermophiles (iii) the number of microbes capable of growth at 80° C. in the sample used to seed the apparatus (the microbial population) was very low and (iv) the types of microbes present in the sample may not be capable of significant growth at 80° C. [typically extremely thermophilic microorganisms are found in hot springs, geysers and deep sea thermal vents (Brock and Madigan, 1988)]. It should be noted that the issues stated above are not a limitation of the method of the invention and in all likeliness could be readily resolved by changing the operating parameters of the method and/or the heterogeneous population of microorganisms used in the method. This example demonstrates that the method can be used to discover microorganisms that grow at high temperatures.

### Example 9 Discovery of Anaerobic Microorganisms

The apparatus described above which contains an oxygen probe, suitable for the measurement of oxygen uptake rate, was limited to the discovery of aerobic (oxygen dependent) microorganisms. The aim of this part of the work was to demonstrate that the method can be used to facilitate the discovery of anaerobic bacteria. The apparatus was therefore modified to enable the isolation of anaerobes with the use of a probe that can detect a molecule used for anaerobic respiration. The ability to use the method for isolation of anaerobic bacteria is valuable because access to other groups of bacteria with potentially different metabolic pathways increases the microbial and enzyme diversity that can be accessed when using the method of the invention.

There are a range of electron acceptors that can be used by anaerobic microorganisms. These include those set out in Table 7:

TABLE 7
Anaerobic respiration processes (Brock and
Madigan, 1988).

| | Types of | Electron | |
|---|---|---|---|
| Respiration | microorganisms | acceptor | Product |
| Sulphur | facultative and | S⁰ | HS— |
| | obligate anaerobic | | |
| | bacteria | | |
| Sulphate | obligate anaerobes | SO₄²⁻ | HS— |
| Carbonate | acetogenic bacteria; | CO₂ | CH₃COO— |
| | obligate anaerobes | | |
| Carbonate | methanogenic | CO₂ | CH₄ |
| | bacteria; obligate | | |
| | anaerobes | | |
| Fumarate | succinogenic bacteria | Fumarate | Succinate |
| Nitrate | facultative anaerobic | NO₃— | NO₂—, N₂O, |
| | bacteria | | N₂ |
| | (denitrification) | | |
| Iron | facultative and | Fe³⁺ | Fe²⁺ |
| | obligate anaerobic | | |
| | bacteria | | |

The apparatus described above was modified to enable the installation of a nitrate ion selective electrode. Nitrate was chosen as the terminal electron acceptor in place of oxygen to demonstrate the discovery of anaerobic bacteria. Denitrification, a process whereby in the absence of oxygen nitrate in used as a terminal electron acceptor and converted into more reduced forms of nitrogen, is quite common (Brock and Madigan, 1988) therefore the presence of microorganisms in the mixed population capable of anaerobic nitrate respiration was considered highly likely. Nitrate was measured continuously using a laboratory bench meter with an analogue output; the data was logged using a computer and simple software developed for this purpose, based on standard data collection techniques.

Nitrate was added to the nutrient feed (the defined medium of Appendix 1) as KNO₃ at a concentration of 1 g l⁻¹. Acetate was used as the test substrate (a carbon source). Acetate was chosen over a fermentable substrate to prevent the growth of fermentative anaerobic microorganisms and the apparatus was sparged with nitrogen to ensure anaerobic conditions were maintained. The acetate was used in an amount of 1.2 g l⁻¹ and the fluid feed rate was 30 ml h⁻¹. The vessel was filled with nutrient medium to establish the response of the nitrate probe to the nitrate that had been added to the nutrient feed.

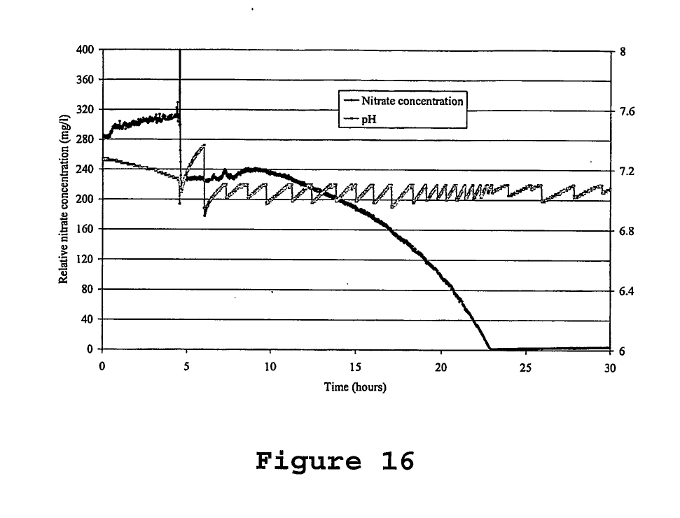

The initial pH was set to pH 7, and the temperature to 30° C. The response of the nitrate probe was fairly stable, increasing gradually from ˜285 to 320 ml l⁻¹ over 4.5 hours. The vessel was then drained to the sample port (a loss of approximately one third of the vessel volume) and refilled with a mixed microbial population (activated sludge) suspended in water. This resulted in partial dilution of the nitrate in the vessel and corresponded to a reduction in the output of the nitrate probe. The apparatus was left in this configuration for 1.3 hours to establish the initial nitrate level. The relative nitrate level after addition of the sludge was stable at 227 mg l⁻¹. The test substrate (acetate) and nutrient pumps were then started at a fluid flow rate of 30 ml h⁻¹. Over the next 17 hours the relative nitrate level decreased to ˜1 mg 1-1. This is illustrated in FIG. 16.

Although optical density could not be used to estimate the biomass concentration due to interference from the initial microbial sample added to the vessel, there are two indicators of microbial activity apparent in the output. The first is the decrease in the nitrate level which is a clear indicator of nitrate consumption and therefore microbial activity. The second indicator is pH. After approximately 17 hours the frequency of the pH oscillations increased. The pH changes are an indicator of substrate consumption (as acetate is consumed the pH increases which is then adjusted by the pH controller to return to pH 7) and the increased requirement for pH control correlated with the maximum rate of nitrate consumption which is indicative of microbial growth. pH control is also shown in FIG. 16.

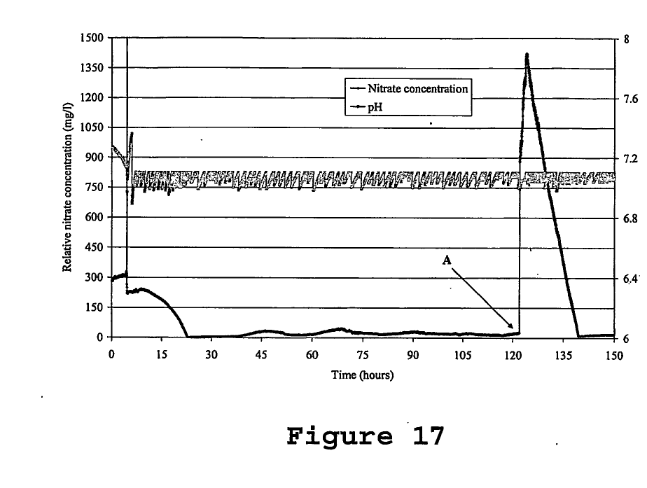

The parameters were not changed for the next 86 hours during which the output of the nitrate probe remained relatively stable with values between 20 and 40 mg l⁻¹ being recorded. The full operation pH and nitrate concentration results are shown in FIG. 17.

To determine whether nitrate or acetate was the limiting nutrient, the test substrate (acetate) feed pump was stopped. If acetate was the limiting nutrient then the nitrate concentration would be expected to rise, the absence of substrate would reduce the energy and therefore, nitrate requirements of the cells. Alternatively, if nitrate is the limiting nutrient the nitrate levels would remain low because the excess acetate would continue to be consumed. After the acetate pump was switched off, no increase in nitrate was observed which suggested that nitrate was the limiting nutrient.

To ensure the nitrate probe was working correctly (not fouled by a biofilm), after 122 hours the vessel was spiked with 5 ml of 218 g l⁻¹ KNO₃. A rapid increase in the output of the nitrate probe was observed indicating the probe was still responding to changes in nitrate concentration. The acetate feed was restarted and the nitrate level again decreased to 20 mg l⁻¹ (See spike in FIG. 17).

To further demonstrate that the nitrate consumption was due to the presence of an active microbial population in the vessel the culture was examined microscopically.

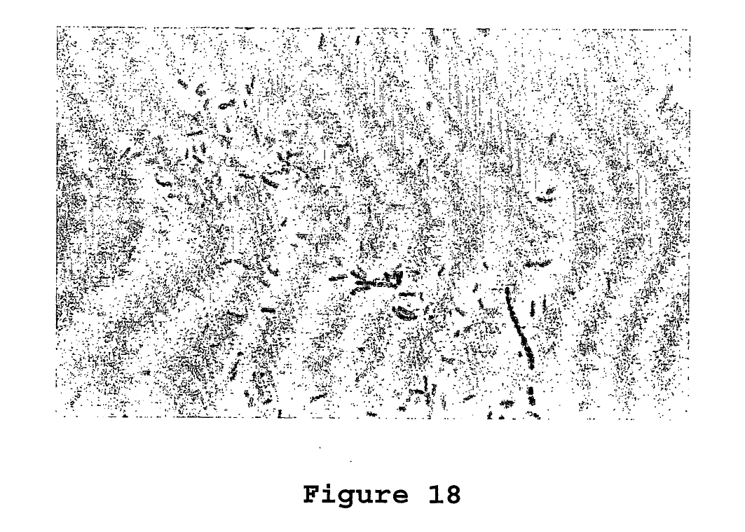

FIG. 18 is a micrograph of the sample. The sample was examined as a wet mount by phase contrast microscopy at a magnification of 1000×. The microbial cells appear as small dark short rods.

The number of cell types present was estimated at being less than ten (not all cell types are apparent in the micrograph) with the dominant types being non-motile rods, motile rods, motile spirals, and filamentous bacteria. This observation showed that a viable population was established by the method under anaerobic conditions and as is observed during aerobic operation, the mixed microbial population that was initially added to the vessel had been sorted into a small or reduced number of microorganisms with the desired properties.

The method was successfully operated under anaerobic conditions and microbial activity was detected by measuring the consumption of nitrate using an ion selective electrode. Although nitrate was the only terminal electron acceptor measured in this example, the system can be easily modified for detection of the other terminal electron acceptors listed in Table 7. The only limitation is the availability of a suitable ion selective electrode. In this example the molecule used for respiration was measured. Electrodes that detect the product(s) of anaerobic respiration could also be used to monitor the microbial discovery process in the method. This example demonstrates that the method can be used to discover anaerobic microorganisms.

Modifications may be made to the preferred embodiments and Examples described above without departing from the spirit and scope of the invention.

### REFERENCES

Bradford, M. (1976). A rapid sensitive method for the quantitation of microgram quantities of protein utilising the principle of protein-dye binding. Analytical Biochemistry 72, 248-254.
Brock, T. D. and Madigan, M. T. (1988). Biology of Microorganisms 5th Edition, Prentice-Hall, New Jersey, USA.
Huang, H., Gong, C. S. and Tsao, G. T. (2002). Production of 1,3-propanediol by Klebsiella pneumoniae; Applied Microbiology Biotechnology 98-100, 687-698.

Nakamura, C. E. and Whited, G. M. (2003). Metabolic engineering for the microbial production of 1,3-propanediol. Current Opinions in Biotechnology 14, 454-459.
Stanier, R. Y., Ingraham, J. L., Wheelis, M, M. L. and Painter, P. R. (1987). General Microbiology 5th Edition, Macmillan Education, London, United Kingdom.

Appendix I

APPENDIX I
Media
Composition of Defined Medium (DM)

| | g l⁻¹ |
|---|---|
| NH₄Cl | 1.0 |
| KH₂PO₄ | 0.5 |
| 10% Na₂SO₄ | 2.0 ml l⁻¹ |
| *MgCl₂•6H₂O | 0.17 |
| *CaCl₂•2H₂O | 0.01 |
| **Trace Metals solution | 1.0 ml l⁻¹ |

All media were made up in reverse osmosis water and adjusted to pH 7.0
with 4M NaOH
All chemicals were of analytical grade.
Where required, media were sterilised by autoclaving at 121° C. for 20
minutes. Large volumes (up to 20 litres) of feed were autoclaved at
121° C. for at least 60 minutes.
*Magnesium and calcium were added as a concentrated sterile stock
solution (17.0 g l⁻¹ MgCl₂•6H₂O; 1.0 g l⁻¹ CaCl₂•2H₂O) after autoclaving
to prevent precipitation with orthophosphate.
Carbon sources were added after the media were autoclaved.
Solid media were prepared by the addition of 15 g l⁻¹ agar.
** The Trace Metals solution contained:

| | g l⁻¹ |
|---|---|
| FeSO₄•7H₂O | 1.0 |
| CoSO₄•7H₂O | 0.2 |
| MnSO₄•H₂O | 0.1 |
| NiCl₂•6H₂O | 0.1 |
| NaMoO₄•2H₂O | 0.05 |
| H₃BO₃ | 0.062 |
| ZnCl₂ | 0.07 |
| CuSO₄•5H₂O | 0.02 |

Composition of Defined Medium (461S) which is a modification of a minimal medium described by Nagel and Andreesen as cited by DSMZ (German culture collection—www dsmz.de/media).

| | ml l⁻¹ |
|---|---|
| *Salts solution | 10 |
| **Trace Elements Stock | 0.7 |
| ***Phosphates | 20 |

* The Salts solution contained:

| | g l⁻¹ |
|---|---|
| CaCl₂ •2H₂O | 1.0 |
| MgSO₂ •7H₂O | 50.0 |
| MnSO₄ | 1.0 |
| NH₄Cl | 30.0 |
| NaCl | 5.0 |

**The chemicals in the Trace Elements Stock were dissolved 5M HCl.
The Trace Elements Stock contained:
(Note: FeSO₄•7H₂O was dissolved in the 5M HCl before the addition
of the other components.)

| | g l⁻¹ (of 5M HCl) |
|---|---|
| FeSO₄•7H₂O | 6.56 |
| ZnCl₂ | 0.14 |
| MnSO₄•H₂O | 0.12 |
| H₃BO₃ | 0.01 |
| CoSO₄•7H₂O | 0.45 |
| CuSO₄•5H₂O | 0.004 |
| NiCl₂•6H₂O | 0.048 |
| NaMoO₄•2H₂O | 0.072 |

***The Phosphates solution contained:

| | g l⁻¹ |
|---|---|
| Na₂HPO₄ | 72.5 |
| KH₂PO₄ | 12.5 |

All media were made up in reverse osmosis water and all chemicals were of analytical grade.

Media were prepared by the mixing the Salts solution and the Trace Elements Stock prior to autoclaving.

Where required, media were sterilised by autoclaving at 121° C. for 20 minutes. Large volumes (up to 20 litres) of feed were autoclaved at 121° C. for at least 60 minutes.

The Phosphates solution was added after autoclaving to prevent precipitation of orthophosphates with the metals in the medium.

Carbon sources were added after the media were autoclaved.

Solid media were prepared by the addition of 15 g l⁻¹ agar.

1-25. (canceled)

26. A method for selectively enriching for a microorganism able to metabolise a test substrate, and/or the enrichment of an enzyme involved in the metabolism of the test substrate, the method comprising the steps of
a) providing a population of microorganisms in a vessel,
b) feeding fluid into the vessel at a controlled flow rate commencing with an initial flow rate, the fluid comprising a nutrient medium and, for at least part of the feed period, the test substrate,
c) producing a signal indicative of the level of a metabolism indicator which is a terminal electron acceptor, over the time-frame of the enrichment, wherein the signal is produced from a probe that takes a reading in the vessel, and
d) providing an output showing the change in level of the metabolism indicator which is based on the signal of the probe to enable assessment of selective enrichment of a microorganism that metabolises the test substrate, and/or the enrichment of an enzyme produced by the microorganism that is involved in the metabolism of the test substrate.

27. The method of claim 26, wherein the output is produced electronically directly from the signal, such that the output is provided on-line.

28. The method of claim 26, wherein the method further comprises presetting conditions to be met by the signal output to result in a change in the fluid flow rate, and changing the flow rate at which fluid is fed into the vessel when the conditions are met, wherein the preset conditions are a combination of a predetermined period of time and a preset value range within which the signal must remain for the predetermined period of time.

29. The method of claim 28, wherein the fluid flow rate is increased when the preset conditions are met.

30. The method of claim 29, wherein the fluid flow rate is increased by proportionally increasing the flow rate of both the nutrient medium and the test substrate.

31. The method of claim 26, wherein the metabolism indicator is the uptake or release of a molecule involved in metabolism of the test substrate.

32. The method of claim 31, wherein the metabolism indicator is selected from the uptake or release of oxygen, carbon dioxide, carbonate, sulphate, sulphur, nitrate, fumarate or iron.

33. The method of claim 31, wherein the metabolism indicator is selected from the uptake or release of oxygen, sulphate, sulphur, nitrate, fumarate or iron.

34. The method of claim 31, wherein the metabolism indicator is the uptake or release of oxygen, and the signal is produced by an oxygen probe.

35. The method of claim 26, wherein the output based on the signal of the level of the metabolism indicator is a visual output.

36. The method of claim 35, wherein the signal of the level of metabolism indicator is provided as a visual output of the metabolism indicator versus time.

37. The method of claim 35, wherein the visual output is updated in periods of duration of 20 minutes or less.

38. The method of claim 35, wherein the visual output is updated in periods of duration of 10 minutes or less.

39. The method of claim 26, wherein the population of microorganisms is a heterogeneous population.

40. The method of claim 39, wherein the population of microorganisms contains at least 10 different species of microorganisms.

41. The method of claim 39, wherein the population of microorganisms is activated sludge.

42. The method of claim 26, wherein the population of microorganisms is homogeneous.

43. The method of claim 26, the method further comprising the step of isolating the enriched microorganism, or isolating the enriched enzyme.

44. The method of claim 26, wherein the test substrate is not a commonly metabolised substrate.

45. The method of claim 26, wherein the test substrate is an organic carbon-containing molecule other than glucose or acetate.

46. The method of claim 26, wherein one, two or all of the conditions in the vessel selected from the pH, temperature and aeration conditions are set by the user prior to commencement of the feeding of test substrate into the vessel.

47. The method of claim 26, wherein a selective pressure is applied to the contents of the vessel to select for a microorganism and/or enzyme that is enriched under the conditions of the selective pressure.

48. The method of claim 47, wherein the selective pressure is selected from one or more of an increase or decrease in pH, temperature, aeration, salt concentration, dissolved gas content, and presence or absence of a chemical compound.

49. The method of claim 47, wherein the microorganism enriched by the method tolerates elevated or reduced temperatures.

50. A microorganism or enzyme enriched or isolated by the method of claim 26.
